(19) United States
(12) Patent Application Publication
(10) Pub. No.: US 2007/0172470 A1
(43) Pub. Date: Jul. 26, 2007

(54) Phosphatase regulation of nucleic acid transcription

(75) Inventors: Gordon N. Gill, La Jolla, CA (US); Michele Yeo, Chapel Hill, NC (US); Patrick S. Lin, Burlingame, CA (US); Michael E. Dahmus, Davis, CA (US)

Correspondence Address: BUCHANAN, INGERSOLL & ROONEY LLP, P.O. BOX 1404, ALEXANDRIA, VA, 22313-1404, US

(73) Assignee: The Regents of the University of California, Oakland, CA (US)

(21) Appl. No.: 10/552,298

(22) PCT Filed: Apr. 1, 2004
(86) PCT No.: PCT/US04/10218

§ 371 Date: Jun. 12, 2006

Related U.S. Application Data
(60) Provisional application No. 60/459,786, filed on Apr. 1, 2003.

(51) Int. Cl. A61K 38/46; C12Q 1/68; C07H 21/04; A61K 48/00; C12N 9/16; C12P 21/06; C12N 15/86; C12N 5/08

(52) U.S. Cl. 424/94.6; 514/44; 435/6; 435/69.1; 435/196; 435/320.1; 435/456; 435/368; 435/325; 536/23.2

(57) ABSTRACT

Nucleic acids, polypeptides and methods are provided for regulating the phosphorylation state of the C-terminal domain (CTD) of RNA polymerase II (RNAP II). Also provided are methods for regulating cell differentiation.

### CROSS REFERENCE TO RELATED APPLICATIONS

This application claims priority from U.S. Provisional Application Ser. No. 60/459,786, filed Apr. 1, 2003, which is incorporated herein by reference.

### STATEMENTS REGARDING FEDERALLY SPONSORED RESEARCH

The invention was funded in part by Grant No. 2RDK13149 awarded by the National Institutes of Health and by Grant No. GM33300 awarded by the National Institutes of Health. The government may have certain rights in the invention.

### TECHNICAL FIELD

This invention relates generally to the regulation of transcription, and more specifically to the regulation of neuronal gene expression.

### BACKGROUND

Protein phosphatases are enzymes that reverse the actions of protein kinases by cleaving phosphate from serine, threonine, and/or tyrosine residues in proteins. Serine/threonine protein phosphatases are associated with the regulation of cellular gene expression, which occurs primarily at the level of transcription initiation by RNA polymerase. Regulated transcription initiation by RNA polymerase (RNAP) II in higher eukaryotes involves the formation of a complex with general transcription factors at promoters. The largest subunit of RNAP II contains a C-terminal domain (CTD) comprised of multiple repeats of the consensus sequence Tyr¹Ser²Pro³Thr⁴Ser⁵Pro⁶Ser⁷. The progression of RNAP II through the transcription cycle is regulated by both the state of CTD phosphorylation and the specific site of phosphorylation within the consensus repeat.

Specific kinases catalyze phosphorylation of Ser 2 and of Ser 5 in the multiple heptad repeats in the CTD of RNAP II. Unphosphorylated RNAP II (RNAP IIA) enters the pre-initation complex where TFIIH catalyzes phosphorylation of Ser 5 to enhance the 7-methy G capping reaction. PTEFb catalyzes phosphorylation of Ser 2, a process necessary for transcript elongation. Phospho RNAP II (RNAP IIO) is ultimately dephosphorylated by FCP1 allowing recycling of the enzyme and re-initiation of transcription.

Mechanisms that regulate the phosphorylation and dephosphorylation of transcription-associated factors can effectively repress and activate transcription of particular genes during. Such mechanisms are particularly important in cellular development and differentiation. Identifying the factors involved in gene activation and repression provides an opportunity to control the differentiation of, for example, stem cells in to specialized cell types.

### SUMMARY

Novel nucleic acid sequences encoding novel small CTD phosphatase (SCP) polypeptides, and dominant-negative mutants thereof, are disclosed. In addition, methods related to identifying substances that modify gene transcription, methods of modifying stem cell differentiation, and methods of treating disease conditions resulting from insufficient, increased or aberrant production of SCP polypeptides are provided. These methods include the use of substances that bind to, or interact with, the SCP proteins, (naturally occurring and biologically active, also referred to herein as wild type SCP proteins) genes encoding the SCP proteins, SCP messenger RNA, or the use of genetically altered SCP proteins.

In one embodiment, isolated nucleic acid molecules are provided. Such nucleic acid molecules include those; 1) consisting of a nucleotide sequence which is at least 80% identical to the nucleotide-sequence of SEQ ID NO:1, 3, 5, 7, 9 or 11; 2) comprising a nucleotide sequence which is at least 80%, 90% or 95% identical to the nucleotide sequence of SEQ ID NO:1, 3, 5, 7, 9 or 11; 3) encoding a polypeptide consisting of the amino acid sequence of SEQ ID NO:2, 4, 6, 8, 10 or 12; 4) encoding a polypeptide comprising the amino acid sequence of SEQ ID NO:2, 4, 6, 8, 10 or 12; 5) encoding a polypeptide comprising the amino acid sequence of SEQ ID NO:2, 4, 6, 8, 10 or 12 with 0 to 50, 0 to 30, or 0 to 10 conservative amino acid substitutions; and 6) encoding a naturally occurring allelic variant of a polypeptide comprising the amino acid sequence of SEQ ID NO:2, 4, 6, 8, 10 or 12, such that the nucleic acid molecule hybridizes to a nucleic acid molecule consisting of SEQ ID NO: 1, 3, 5, 7, 9 or 11, or a complement thereof, under stringent conditions.

In another embodiment, isolated nucleic acid molecules deposited as ATCC Accession Numbers BE300370, AL520011, and AL520463, or a complement thereof, are provided.

In other embodiments nucleic acid molecules comprising the nucleotide sequence of SEQ ID NO:1, 3, 5, 7, 9 or 11 or consisting of t he nucleotide sequence of SEQ ID NO:1, 3, 5, 7, 9 or 11, are provided.

Also provided are vectors containing the nucleic acid molecules disclosed herein and host cells containing such vectors. Methods of producing a polypeptide by culturing such host cells are also provided.

In yet another embodiment, isolated polypeptides are provided. Such polypeptides include a polypeptide: 1) consisting of an amino acid sequence which is at least 80%, 90% or 95% identical to the amino acid sequence of SEQ ID NO:2, 4, 6, 8, 10 or 12; 2).comprising an amino acid sequence which is at least 80% identical to the amino acid sequence of SEQ ID NO:2, 4, 6, 8, 10 or 1; 3) comprising the amino acid sequence of SEQ ID NO:2, 4, 6, 8, 10 or 12 with 0 to 50, 0 to 30 or 0 to 10 conservative amino acid substitutions; 4) encoded by a nucleic acid molecule comprising a nucleotide sequence which is at least 80%, 90% or 95% identical to a nucleic acid comprising the nucleotide sequence of SEQ ID NO:1, 3, 5, 7, 9 or 11; and 5) that are naturally occurring allelic variants of a polypeptide comprising the amino acid sequence of SEQ ID NO:2, 4, 6 or 8, such that the polypeptide is encoded by a nucleic acid molecule which hybridizes to a nucleic acid molecule consisting of SEQ ID NO: 1, 3, 5, 7, 9 or 11, or a complement thereof, under stringent conditions.

Also included are polypeptides comprising the amino acid sequence of SEQ ID NO:2, 4, 6, 8, 10 or 12 and polypeptides consisting of the amino acid sequence of SEQ ID NO:2, 4, 6, 8, 10 or 12. Such polypeptides are generally phosphatases or a phosphatase inactive mutant. The phosphatase is generally a serine phosphatase that dephosphorylates serine 5 within the C-terminal binding domain (CTD) of RNA polymerase II. The phosphatase can be small CTD phosphatase-1 (SCP1), small CTD phosphatase-2 (SCP2), or small CTD phosphatase-3 (SCP3).

Also provided are antibodies that selectively bind to a polypeptides provided herein.

In another embodiment, methods of promoting differentiation of a non-neuronal cell in to a cell of the nervous system are provided. Such methods include contacting the non-neuronal cell with a nucleic acid molecule comprising a nucleic acid sequence encoding a polypeptide selected from the group consisting of SEQ ID NO:10 and SEQ ID NO:12 and expressing the polypeptide in the cell such that the dominant-negative SCP mutant inhibits the activity of endogenous SCP. Such methods are useful for promoting, for example, differentiation of stem cells in to nerve tissue including, but not limited to, neurons, sensory neurons, motoneurons, interneurons, glial cells, microglial cells and astrocytes.

In another embodiment, a method of inhibiting differentiation of a non-neuronal cell in to a cell of the nervous system by contacting the cell with a nucleic acid molecule including a nucleic acid sequence encoding a polypeptide selected from SEQ ID NO:2. SEQ ID NO:4, SEQ ID NO:6 and SEQ ID NO:8, and expressing the polypeptide in the cell, is provided.

Also provided are methods of promoting RNA polymerase II associated transcription in a cell by contacting the cell with a nucleic acid molecule including a nucleic acid sequence encoding a polypeptide selected from SEQ ID NO:10 and SEQ ID NO:12, and expressing the polypeptide in the cell.

In another embodiment, a composition that includes an inhibitor of small CTD phosphatase (SCP) gene expression is provided. The inhibitor can be a small molecule inhibitor of gene expression, an anti-sense oligonucleotide, or a small interfering RNA molecule (siRNA or RNAi). The inhibitor of SCP gene expression can, for example, specifically bind to a polynucleotide that includes: 1) a sequence selected from the group consisting of SEQ ID NO:1, 3, 5 and 7; 2) a complement of a polynucleotide comprising a sequence selected from the group consisting of SEQ ID NO:1, 3, 5 and 7; 3) a reverse sequence of a polynucleotide comprising a sequence selected from the group consisting of SEQ ID NO:1, 3, 5 and 7; 4) a polynucleotide that encodes a polypeptide comprising a sequence selected from the group consisting of SEQ ID NO:2, 4, 6 and 8; 5) a complement of a polynucleotide that encodes a polypeptide comprising a sequence selected from the group consisting of SEQ ID NO:2, 4, 6 and 8; or 6) a reverse sequence of a polynucleotide that encodes a polypeptide comprising a sequence selected from the group consisting of: SEQ ID NO:2, 4, 6 and 8.

In another embodiment, a method of promoting the differentiation of a non-neuronal cell in to a cell of the nervous system is provided. Such methods can be accomplished by contacting the non-neuronal cell with the composition described above in a sufficient concentration to inhibit the expression of a small CTD phosphatase (SCP).

In yet another embodiment, a method for identifying a compound that modulates the activity of an SCP polypeptide is provided. The method includes contacting an SCP polypeptide provided herein with a test compound and determining the effect of the test compound on the activity of the polypeptide to thereby identify a compound which modulates the activity of the polypeptide.

In another embodiment, a method of modulating the differentiation of a mammalian stem cell by contacting the stem cell with a compound that modulates SCP1, SCP2 or SCP3 activity, under conditions suitable for differentiation of said stem cell, is provided.

In another embodiment, a method of transplanting a mammalian stem cell or progenitor cell to a patient in need thereof including (a) contacting the stem cell or progenitor cell with a compound that inhibits SCP1, SCP2 or SCP3 activity to produce a treated stem cell or progenitor cell; and (b) transplanting the treated stem cell into said. patient, is provided.

### BRIEF DESCRIPTION OF THE DRAWINGS

FIG. 1A depicts a sequence alignment of amino acid sequences surrounding the catalytic domain and relation of SCP to FCP1.

FIG. 1B depicts the domain structures of FCP1 and SCP polypeptides.

FIG. 2A is a graph depicting phosphatase activity of SCP1 at various pH.

FIG. 2B is a graph depicting the divalent metal ion requirements for SCPL phosphatase activity.

FIG. 2C is an autoradiogram depicting a CTD phosphatase assay of FCP1 on GST-CTDo and RNAP IIO prepared by MAPK2/ERK2.

FIG. 2D is an autoradiogram depicting a CTD phosphatase assay of SCP1 on GST-CTDo and RNAP IIO prepared by MAPK2/ERK2.

FIG. 3A is an autoradiogram and graph depicting substrate specificity of SCP1 for dephosphorylation of RNAP IIO prepared with various CTD kinases.

FIG. 3B is a graph depicting the effects of GST-SCP1 214 on a 28 aa peptide consisting of heptad repeats containing either Ser 5 phosphate or Ser 2 phosphate.

FIG. 4 is an autoradiogram depicting the effect of RAP74 on CTD phosphatase activity of SCP1 and SCP2.

FIG. 5A depicts nuclear localization and association of SCP1 with RNAP II. Cells were co-stained for the endosomal marker EEAL using mouse anti-EEA1 and Alexa Fluor 594 conjugated goat anti-mouse (red). Nuclei were detected with DAPI (blue).

FIG. 5B depicts immunofluorescence microscopy detection of endogenous SCP1 using rabbit polyclonal IgG 6307 and Alexa Fluor 488 conjugated goat anti-rabbit IgG (green).

FIG. 5C is a gel depicting co-immunoprecipitation of RNAP II and endogenous SCP1.

FIG. 6A is a graph depicting the effects of targeted SCP1 261 on reporter gene expression.

FIG. 6B is a graph depicting the effects of SCP1 261 and mutant SCP1 261 on basal promoter activity.

FIG. 6C is a graph depicting the differing effects of SCP1 261 and phosphatase-inactive SCP1 261 on Gal 4-VP16 stimulated gene expression.

FIG. 6D is a graph depicting the effects of SCP1 261 and phosphatase-inactive SCP1 261 on ligand activated receptor activity.

FIG. 6E is a graph depicting competitive effects of mutant SCP1 261 with SCP1 261.

FIG. 7A depicts Northern blot analysis of the expression of SCP1 in human tissues.

FIG. 7B depicts in situ hybridization analysis of expression of SCP1 in e 10.5 mouse cervical spinal cord.

FIG. 7C depicts in situ analysis of the expression of isl-1 in areas of the developing spinal cord where SCP1 is not expressed.

FIG. 8A depicts co-immunoprecipitation of SCP1 and REST/NRSF.

FIG. 8B depicts chromatin immunoprecipitation using anti-SCP antibody.

FIG. 9A depicts undifferentiated P19 cells.

FIG. 9B depicts P19 cells differentiated into neuron like cells (NLC) by treatment with retinoic acid and growth in selective medium.

FIG. 9C depicts differentiated GFP expressing P19 cells.

FIG. 9D depicts differentiated mutant SCP1-expressing P19 cells.

FIG. 9E depicts differentiated SCP1-expressing P10 cells.

FIG. 9F depicts differentiated REST/NRSF-expressing P19 cells.

FIG. 10 depicts the quantitation of transcripts using real time quantitative RT-PCR and the effect of siRNA on the transcript quantity.

### DETAILED DESCRIPTION

Novel small CTD phopsphatases (SCP's) polypeptides, and nucleic acid molecules encoding such polypeptides, are provide. Also provided are methods of modifying gene transcription by modulating the expression and/or activity of SCP's. Such methods can be used to inhibit or promote differentiation of cells. Also provided are methods for identifying nucleic acids compounds that bind to, or interact with, one or more SCP proteins or the DNA/RNA encoding the SCP proteins and, thus, modifying the activity of an SCP protein on RNA polymerase II, or on other transcription factors essential to gene transcription.

Compounds that bind to, or interact with, one, or more SCP proteins or the DNA/RNA encoding the proteins can inhibit or enhance the activity of the RNA polymerase II, thus, inhibiting or enhancing gene transcription. For example, antisense, nonsense or interfering (i.e., RNAi) nucleotide sequences that modulate SCP translation or transcription can effect the RNA polymerase II-mediated gene transcription.

Unphosphorylated RNAP II, designated RNAP IIA, enters the pre-initiation complex where phosphorylation of Ser 5 is catalyzed by TFIIH (which contains cdk7/cyclin H subunits) concomitant with transcript initiation. This generates the phosphorylated form of RNAP II, designated RNAP IIO. Ser 5 phosphorylation facilitates the recruitment of the 7-methyl G capping enzyme complex (Cho et al., (1997) Genes Dev. 11:3319-3326; McCracken et al., (1997) Genes Dev. 11:3306-3318). Phosphorylation of Ser 2, is catalyzed by the cyclin-dependent kinase P-TEFb (which contains cdk9/cyclin T subunits). During transcript elongation in yeast there is extensive turnover of Ser 2 phosphates mediated by FCP1 and Ctk1, the putative PTEFb homolog (Cho et al., (2001) Genes Dev. 15:3319-3329). Finally, dephosphorylation of Ser 2 by the FCP1 phosphatase regenerates RNAP IIA thereby completing the cycle.

FCP1 is a class C (PPM) phosphatase containing a BRCT domain that is required for interaction with RNAP II and dephosphorylation of the CTD (Cho et al, (1999) Genes Dev. 13:1540-1552; Archambault et al., (1997) Proc. Natl. Acad. Sci. USA 94:14300-14305). FCP1 interacts with and is stimulated by RAP74, the larger subunit of TFIIF. Class C phosphatases are resistant to inhibitors that block other classes of Ser/Thr phosphatases and bind Mg2+ or Mn2+ in the binuclear metal center of the catalytic site. The ψψψDXDX(T/V)ψψ motif (where ψ=hydrophobic residue) present in the FCP1 homology domain characterizes a subfamily of class C phosphatases with both Asp residues being essential for activity.

Synthetic lethality is observed between mutant FCP1 and reduced levels of RNAP II in S. cerevisiae and S. pombe, indicating that FCP1 is an essential gene (Kobor et al., (1999) Mol. Cell 4:55-62; Kimura et al., (2002) Mol. Cell Biol. 22:1577-1588). Mammalian FCP1 dephosphorylates both Ser 2 and Ser 5 in vitro in the context of native RNAP II.

Although FCP1 is the only reported CTD phosphatase, examination of the databases reveals additional genes that consist principally of a domain with homology to the CTD phosphatase domain of FCP1. Three closely related human genes encoding small proteins with CTD phosphatase domain homology, but lacking a BRCT domain, have been identified. In the present study we show that a gene located on chromosome 2 encodes a nuclear CTD phosphatase. This protein preferentially dephosphorylates Ser 5 within the CTD of RNAP II and is stimulated by RAP74. Expression of this small CTD phosphatase (SCP1) inhibits activated transcription from a variety of promoter-reporter gene constructs whereas expression of a mutant lacking phosphatase activity enhances transcription. These novel small CTD phosphatase are involved in the regulation of RNAP II transcription.

The present invention is based, at least in part, on the discovery of novel molecules, referred to herein as SCP protein and nucleic acid molecules, which comprise a family of molecules having certain conserved structural and functional features. The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally occurring and can be from either the same or different species. For example, a family can contain a first protein of human origin, as well as other, distinct proteins of human origin or alternatively, can contain homologues of non-human origin. Members of a family may. also have common functional characteristics associated with serine phosphatase activity, and particularly with dephosphorylation of the CTD of RNA Polymerase II.

In the vertebrate spinal cord expression of a temporally ordered sequence of transcriptional repressors and activators direct formation of glia and specialized neuronal cell types from common precursors (Jessell, Nature Rev Genetics (2000) 1:20-29.). Repressor systems also direct long term silencing of inappropriate gene expression in differentiated cells. A 23 bp DNA element (repressor element 1 (RE-1)) binds the Zn²⁺-finger-containing protein REST (RE-1 silencing transcription factor)/NRSF (Neuron-Restrictive Silencer Factor) (Chong, et al., Cell (1995) 80:949-957; Schoenherr and Anderson. Science (1995) 267:1360-1363; Chen, et al., Nature Genetics (1998) 20:136-142.). REST/NRSF recruits a multiprotein complex that acts to repress gene transcription via histone deacetylation and via methylation of DNA and of histone H3 (Naruse, et al. Proc. Natl. Acad. Sci USA (1999) 96:1369-13696; Hakimi, et al. Proc. Natl. Acad. Sci. USA (2002) 99:7420-7425; Kokwra, et al. J. Biol. Chem. (2001) 276:34115-34121). These defined mechanisms of gene silencing via REST/NRSF result from covalent modifications of chromatin.

The present studies further identify SCPs as functional components of the REST/NRSF silencing complex. Because phosphorylation of serine 5 of the CTD of RNAP II is essential for initiation of transcription, dephosphorylation is an effective and reversible mechanism to inhibit transcription. SCPs repress transcription of a variety of regulated reporter genes but in vivo are specifically recruited to RE-1 elements in neuronal genes. Other genes that contain an FCP1 phosphatase homology domain defined by the YYY DX(T/V) YY (where Y represents a hydrophobic residue) sequence (Kang and Dahmus, (1993) J. Biol. Chem. 268:25033-25040; Chambers and Dahmus, (1994) J. Biol. Chem. 269:26243-26248) are candidates to function in regulating other classes of genes.

As P19 stem cells differentiate into neurons both SCPs and REST/NRSF expression are silenced indicating that silencers of neuronal silencers (SONS) exist. Inhibition of SCP by a dominant negative form of SCP in replicating P19 cells increases the fraction of stem cells that morphologically develop into neurons indicating that the same mechanisms that silence neuronal gene expression in differentiated non-neuronal cells act in neuronal stem cells.

In addition, an siRNA-mediated decrease in the Drosophila SCP gene product on neuronal gene expression in S2 cells was also identified, further indicating that SCP is part of REST/NRSF complexes and functions in silencing neuronal gene expression. Dephosphorylation of Ser 5 of the CTD of RNAP II is a target for reversible mechanisms involved in the inhibition of neuronal gene transcription using, for example, siRNA.

Nucleic Acids and Polypeptides

The SCP proteins, fragments thereof, and derivatives and other variants of the sequence in SEQ ID NO:2, 4, 6, 8, 10 or 12, thereof are collectively referred to as “polypeptides or proteins of the invention” or SCP “polypeptides or proteins”. Nucleic acid molecules encoding such polypeptides or proteins are collectively referred to as “nucleic acids of the invention” or “SCP nucleic acids.” SCP molecules refer to SCP nucleic acids, polypeptides, and antibodies.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions are hybridization in 6× sodium chloride/sodium citrate (SSC) at about 45° C., followed by one or more washes in 0.2×SSC, 0.1% SDS at 60° C. Preferably, stringent hybridization conditions are hybridization in 6× sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied) are 0.5M Sodium Phosphate, 7% SDS at 65° C., followed by one or more washes at 0.2×SSC, 1% SDS at 65° C. For example, an isolated nucleic acid molecule of the invention that hybridizes under stringent conditions to the sequence of SEQ ID NO:1, 3, 5 or 7 corresponds to a naturally occurring nucleic acid molecule.

The definitions of the terms “complement”, “reverse complement” and “reverse sequence”, as used herein, are best illustrated by the following example. For the sequence 5′ AGGACC 3′, the complement, reverse complement and reverse sequence are as follows:

| | |
|---|---|
| complement: | 3′ TCCTGG 5′ |
| reverse complement: | 3′ GGTCCT 5′ |
| reverse sequence: | 5′ CCAGGA 3′. |

Preferably, sequences that are complements of a specifically recited polynucleotide sequence are complementary over the entire length of the specific polynucleotide sequence.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a SCP protein, preferably a mammalian SCP protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of SCP protein having less than about 30%, 20%, 10% and more preferably 5% (by dry weight), of non-SCP protein (also referred to herein as a “contaminating protein”), or of chemical precursors or non-SCP chemicals. When the SCP protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of SCP1, SCP2 or SCP3 (e.g., the sequence of SEQ ID NO:1, 3, or 5 or the nucleotide sequence of the DNA insert of the plasmid deposited with ATCC as Accession Numbers BE300370, AL520011, or AL520463,) without abolishing or more preferably, without substantially altering a biological activity, whereas an “essential” amino acid residue results in such a change. For example, amino acid residues that are conserved among the polypeptides of the present invention are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a SCP protein is preferably replaced with another amino acid residue from the same side chain family. Alternatively, in another embodiment, mutations can be introduced randomly along all or part of a SCP coding sequence, such as by saturation mutagenesis, and the resultant mutants can be screened for SCP biological activity to identify mutants that retain activity. Following mutagenesis of SEQ ID NO:1 or 8, the resulting dominant-negative mutants have a sequence set forth in SEQ ID NO:10 or 12, respectively.

As used herein, a “biologically active portion” of a SCP protein includes a fragment of a SCP protein-which participates in an interaction between a SCP molecule and a non-SCP molecule, e.g. RNA polymerase II or REST/NRSF. Biologically active portions of a SCP protein include peptides comprising amino acid sequences sufficiently homologous to or derived from the amino acid sequence of the SCP protein, e.g., the amino acid sequence shown in SEQ ID NO:2, 4 or 6, which include less amino acids than the full length SCP proteins, and exhibit at least one activity of a SCP protein. Typically, biologically active portions comprise a domain or motif with at least one activity of the SCP protein, e.g., dephosphorylation of RNA polymerase II or interacting with REST/NRSF. A biologically active portion of a SCP protein can be a polypeptide which is, for example, 10, 25, 50, 100, 200 or more amino acids in length. Biologically active portions of a SCP protein can be used as targets for developing agents which modulate a SCP mediated activity, e.g., the regulation of differentiation of a non-neuronal cell in to a neuronal cell.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., SEQ ID NO:2, 4 or 6). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available on the world wide web at gcg.com), using a NWSgapdna.CMP matrix and a gap weight of 40, 50, 60, 70, or 80 and a length weight of 1, 2, 3, 4, 5, or 6.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to SCP nucleic acid molecules of the invention. BLAST protein searches can be performed with the XBLAST program, score=50, wordlength=3 to obtain amino acid sequences homologous to SCP protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the world wide web at ncbi.nlm.nih.gov.

Isolated Nucleic Acid Molecules

In one aspect, the invention provides, an isolated or purified, nucleic acid molecules that encode SCP polypeptides described herein, e.g., a full length SCP1, SCP2, SCP3 or SCP1 214 protein or a fragment thereof, e.g., a biologically active portion of an SCP protein. Also included is a nucleic acid fragment suitable for use as a hybridization probe, which can be used, e.g., to a identify nucleic acid molecule encoding a polypeptide of the invention, SCP mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, 3, 5 or 7, or mutant derivatives thereof including SEQ ID NO:9 or 11. In one embodiment, the nucleic acid molecule includes sequences encoding the human SCP1, SCP2, or SCP3 protein (i.e., “the coding region”, from nucleotides 1-781 SEQ ID NO:1; nucleotides 1-852 SEQ ID NO:3; nucleotides 1-798 SEQ ID NO:3), as well as 5′ untranslated sequences. In another embodiment, the nucleic acid molecule encodes a sequence corresponding to the mature protein of SEQ ID NO:2, 4, 6 or 8.

In another embodiment, an isolated nucleic acid molecule of the invention includes a nucleic acid molecule which is a complement of the nucleotide sequence shown in SEQ ID NO:1, 3, 5, or 7, or a portion of any of these nucleotide sequences. In other embodiments, the nucleic acid molecule of the invention is sufficiently complementary to the nucleotide sequence shown in SEQ ID NO:1, 3, 5 or 7 such that it can hybridize to the nucleotide sequence shown in SEQ ID NO:1, 3, 5 or 7, thereby forming a stable duplex.

In one embodiment, an isolated nucleic acid molecule of the present invention includes a nucleotide sequence which is at least about 50%, 55%, 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:1, 3, 5 or 7, or a portion, preferably of the same length, of any of these nucleotide sequences.

SCP Nucleic Acid Variants

The invention further encompasses nucleic acid molecules that differ from the nucleotide sequence shown in SEQ ID NO:1, 3, 5 or 7, or the dominant negative SCP mutants provided in SEQ ID NO:9 and 11. Such differences can be due to degeneracy of the genetic code (and result in a nucleic acid which encodes the same SCP proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, 4, 6, or 8. If alignment is needed for this comparison the sequences should be aligned for maximum homology. “Looped” out sequences from deletions or insertions, or mismatches, are considered differences.

Nucleic acids of the invention can be chosen for having codons, which are preferred, or npn-preferred, for a particular expression system. E.g., the nucleic acid can be one in which at least one colon, at preferably at least 10%, or 20% of the codons has been altered such that the sequence is optimized for expression in e. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO: 1, 3, 5 or 7, e.g., as follows: by at least one but less than 10, 20, 30, or 40 nucleotides; at least one but less than 1%, 5%, 10% or 20% of the in the subject nucleic acid. If necessary for this analysis the sequences should be aligned for maximum homology.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:2, 4, 6, or 8, or a fragment of this sequence. Such nucleic acid molecules can readily be identified as being able to hybridize under stringent conditions, to the nucleotide sequence shown in SEQ ID NO:2, 4, 6, or 8 or a fragment of the sequence. Nucleic acid molecules corresponding to orthologs, homologs, and allelic variants of the SCP cDNAs of the invention can further be isolated by mapping to the same chromosome or locus as the SCP gene.

Preferred variants include those that are correlated with dephosphorylation of RNA polymerase II, for example.

Allelic variants of SCP, e.g., human SCP, include both functional and non-functional proteins. Functional allelic variants are naturally occurring amino acid sequence variants of the SCP protein within a population that maintain the ability to dephosphorylate RNA polymerase II. Functional allelic variants will typically contain only conservative substitution of one or more amino acids of SEQ ID NO:2, 4, 6, or 8, or substitution, deletion or insertion of non-critical residues in non-critical regions of the protein. Non-functional allelic variants are naturally-occurring amino acid sequence variants of the SCP, e.g., human SCP, protein within a population that do not have the ability to dephosphorylate RNA polymerase II. Non-functional allelic variants will typically contain a non-conservative substitution, a deletion, or insertion, or premature truncation of the amino acid sequence of SEQ ID NO:2, 4, 6 or 8, or a substitution, insertion, or deletion in critical residues or critical regions of the protein.

Moreover, nucleic acid molecules encoding other SCP family members and, thus, which have a nucleotide sequence which differs from the SCP sequences of SEQ ID NO:1, 3, 5 or 7, are intended to be within the scope of the invention.

Antisense Nucleic Acid Molecules, Ribozymes, Modified SCP Nucleic Acid Molecules and siRNA

In another embodiment, isolated SCP nucleic acid molecules which are antisense to SCP are provided. Such molecules can be used to inhibit the expression of SCP in a cell, for example a stem cell, such that the cell can differentiate in to a cell of the nervous system. As used herein “cell” is used in its usual biological sense, and does not refer to an entire multicellular organism, e.g., specifically does not refer to a human. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell.

“Cells of the nervous system” refers to cells that are specifically related to the nervous system of an animal. For example, a “cell of the nervous system” can be a “neuron” or a “nerve cell”, which is an excitable cell specialized for the transmission of electrical signals over long distances. Neurons receive input from sensory cells or other neurons and send output to muscles or other neurons. Exemplary “neurons” include a “sensory neuron” that has sensory input, a “motoneuron” that has muscle outputs, or “interneuron” that connects only with other neurons. A “cell of the nervous system” can also be a specialized non-neuronal nervous cell, for example a glial cell, which is a cell that surrounds a neuron, providing mechanical and physical support and electrical insulation between neurons. Examples of glial cells include, but are not limited to, microglial cells and astrocytes.

An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire SCP coding strand, or to only a portion thereof (e.g., the coding region of human SCP1, SCP2, SCP3 or SCP1 215, corresponding to SEQ ID NO:1, 3, 5, and 7, respectively). In another embodiment, the antisense nucleic acid molecule is antisense to a “noncoding region” of the coding strand of a nucleotide sequence encoding SCP (e.g., the 5′ and 3′ untranslated regions).

An antisense nucleic acid can be designed such that it is complementary to the entire coding region of SCP mRNA, but more preferably is an oligonucleotide which is antisense to only a portion of the coding or noncoding region of SCP mRNA. For example, the antisense oligonucleotide can be complementary to the region surrounding the translation start site of SCP mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used: The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a SCP protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic. acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a SCP-encoding nucleic acid can include one or more sequences complementary to the nucleotide sequence of a SCP cDNA disclosed herein (i.e., SEQ ID NO:1, 3, 5, or 7), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a SCP-encoding mRNA. See, e.g., Cech et al. U.S. Pat. No. 4,987,071; and Cech et al. U.S. Pat. No. 5,116,742. Alternatively, SCP mRNA can be used to select a catalytic RNA having a specific ribonuclease activity from a pool of RNA molecules. See, e.g., Bartel, D. and Szostak, J. W. (1993) Science 261:1411-1418.

SCP gene expression can be inhibited by targeting nucleotide sequences complementary to the regulatory region of the SCP (e.g., the SCP promoter and/or enhancers) to form triple helical structures that prevent transcription of the SCP gene in target cells. See generally, Helene, C. (1991) Anticancer Drug Des. 6(6):569-84; Helene, C. et al. (1992) Ann. N. Y. Acad. Sci. 660:27-36; and Maher, L. J. (1992) Bioassays 14(12):807-15. The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

An SCP nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra, Perry-O'Keefe et al. Proc. Natl. Acad. Sci. 93: 14670-675.

PNAs of SCP nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of SCP nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, isolated siRNA nucleic acid molecules which destabilize SCP transcripts are provided. Such molecules can be used to inhibit the expression of SCP in a cell, for example a stem cell, such that the cell can differentiate in to a cell of the nervous system.

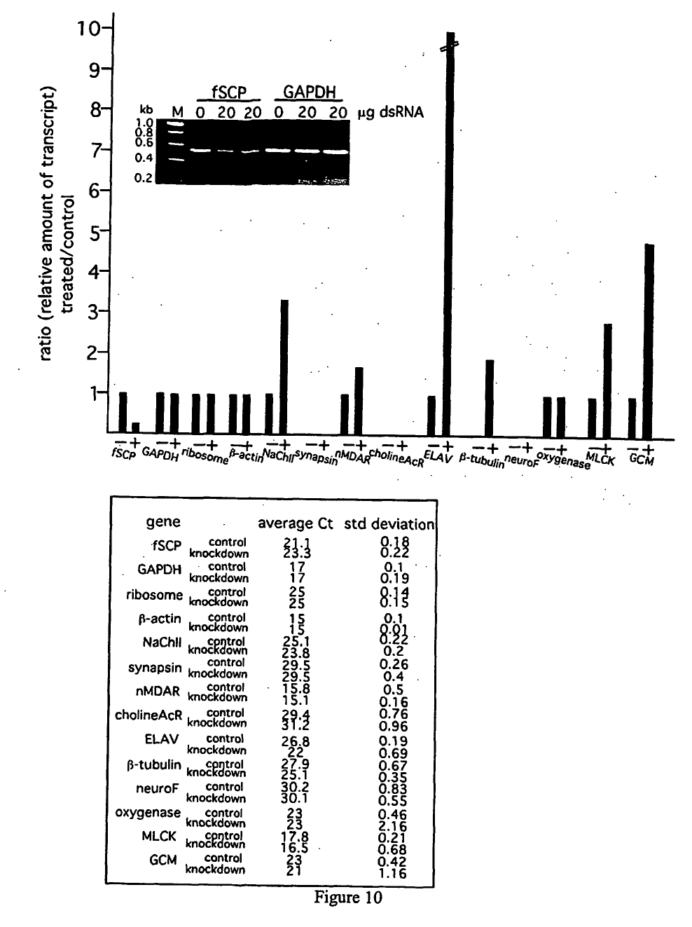

The identification of potential siRNA target sites in an SCP RNA sequence can be identified using the information provided herein. For example, the inventors have utilized siRNA in a Drosophila system to inhibit the expression of Drosophila SCP (e.g., see FIG. 10). This information, coupled with the knowledge of the skilled artisan, can be used to generate siRNA suitable for use in other cell types, such as mammalian stem cells.

To identify sites in a target transcript (i.e., SCP RNA), the sequence of an RNA target of interest is screened for target sites, for example by using a computer folding algorithm. In a non-limiting example, the sequence of a gene or RNA gene transcript derived from a database, such as Genbank, is used to generate siRNA targets having complimentarily to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well. Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. In a non-limiting example, anywhere from 1 to 1000 target sites are chosen within the transcript based on the size of the siRNA construct to be used. High throughput screening assays can be developed for screening siRNA molecules using methods known in the art, such as with multi-well or multi-plate assays to determine efficient reduction in target gene expression.

The following non-limiting steps can be used to carry out the selection of siRNAs targeting a given gene sequence or transcript.

The target sequence is parsed in silico into a list of all fragments or subsequences of a particular length contained within the target sequence. This step is typically carried out using a custom Perl script, but commercial sequence analysis programs such as Oligo, MacVector, or the GCG Wisconsin Package can be employed as well.

In some instances the siRNAs correspond to more than one target sequence; such would be the case for example in targeting many different strains of a viral sequence, for targeting different transcripts of the same gene, targeting different transcripts of more than one gene, or for targeting both the human gene and an animal homolog. In this case, a subsequence list of a particular length is generated for each of the targets, and then the lists are compared to find matching sequences in each list. The subsequences are then ranked according to the number of target sequences that contain the given subsequence; the goal is to find subsequences that are present in most or all of the target sequences. Alternately, the ranking can identify subsequences that are unique to a target sequence, such as a mutant target sequence. Such an approach would enable the use of siRNA to target specifically the mutant sequence and not effect the expression of the normal sequence.

In some instances the siRNA subsequences are absent in one or more sequences while present in the desired target sequence; such would be the case if the siRNA targets a gene with a paralogous family member that is to remain untargeted. A subsequence list of a particular length is generated for each of the targets, and then the lists are compared to find sequences that are present in the target gene but are absent in the untargeted paralog.

The ranked siRNA subsequences can be further analyzed and ranked according to GC content. A preference can be given to sites containing 30-70% GC, with a further preference to sites containing 40-60% GC.

The ranked siRNA subsequences can be further analyzed and ranked according to self-folding and internal hairpins. Weaker internal folds are preferred; strong hairpin structures are to be avoided.

The ranked siRNA subsequences can be further analyzed and ranked according to whether they have runs of GGG or CCC in the sequence. GGG (or even more Gs) in either strand can make oligonucleotide synthesis problematic, so it is avoided whenever better sequences are available. CCC is searched in the target strand because that will place GGG in the antisense strand.

The ranked siRNA subsequences can be further analyzed and ranked according to whether they have the dinucleotide UU (uridine dinucleotide) on the 3′ end of the sequence, and/or AA on the 5′ end of the sequence (to yield 3′ UU on the antisense sequence). These sequences allow one to design siRNA molecules with terminal TT thymidine dinucleotides.

Four or five target sites are chosen from the ranked list of subsequences as described above. For example, in subsequences having 23 nucleotides, the right 21 nucleotides of each chosen 23-mer subsequence are then designed and synthesized for the upper (sense) strand of the siRNA duplex, while the reverse complement of the left 21 nucleotides of each chosen 23-mer subsequence are then designed and synthesized for the lower (antisense) strand of the siRNA duplex. If terminal TT residues are desired for the sequence then the two 3′ terminal nucleotides of both the sense and antisense strands are replaced by TT prior to synthesizing the oligos.

The siRNA molecules are screened in an in vitro, cell culture or animal model system to identify the most active siRNA molecule or the most preferred target site within the target RNA sequence.

The siRNA molecules of the invention can be designed to inhibit SCP gene expression through RNAi targeting of a variety of RNA molecules. In one embodiment, the siRNA molecules of the invention are used to target various RNAs corresponding to a target gene. Non-limiting examples of such RNAs include messenger RNA (mRNA), alternate RNA splice variants of target gene(s), post-transcriptionally modified RNA of target gene(s), pre-mRNA of target gene(s), and/or RNA templates used for SCP activity. If alternate splicing produces a family of transcripts that are distinguished by usage of appropriate exons, the instant invention can be used to inhibit gene expression through the appropriate exons to specifically inhibit or to distinguish among the functions of gene family members. Non-limiting examples of applications of the invention relating to targeting these RNA molecules include therapeutic pharmaceutical applications, pharmaceutical discovery applications, molecular diagnostic and gene function applications, and gene mapping.

In another embodiment, the siRNA molecules of the invention are used to target conserved sequences corresponding to a gene family or gene families such as SCP genes. As such, siRNA molecules targeting multiple SCP targets can provide increased therapeutic effect. In addition, siRNA can be used to characterize pathways of gene function in a variety of applications. For example, the present invention can be used to inhibit the activity of target gene(s) in a pathway to determine the function of uncharacterized gene(s) in gene function analysis, MRNA function analysis, or translational analysis. The invention can be used to determine potential target gene pathways involved in various diseases and conditions toward pharmaceutical development. The invention can be used to understand pathways of gene expression involved in development, such as prenatal development, postnatal development and/or aging.

In one embodiment, the invention features a method comprising: (a) analyzing the sequence of a RNA target encoded by an SCP gene; (b) synthesizing one or more sets of siRNA molecules having sequence complementary to one or more regions of the RNA of (a); and (c) assaying the siRNA molecules of (b) under conditions suitable to determine RNAi targets within the target RNA sequence. In another embodiment, the siRNA molecules of (b) have strands of a fixed length, for example about 23 nucleotides in length. In yet another embodiment, the siRNA molecules of (b) are of differing length, for example having strands of about 19 to about 25 (e.g., about 19, 20, 21, 22, 23, 24, or 25) nucleotides in length.

Isolated SCP Polypeptides

In another embodiment, an isolated SCP protein, or fragment, e.g., a biologically active portion, for use as immunogens or antigens to raise or test (or more generally to bind) anti-SCP antibodies are provided. SCP protein or fragments thereof can be produced by recombinant DNA techniques or synthesized chemically.

Polypeptides of the invention include those which arise as a result of the existence of multiple genes, alternative transcription events, alternative RNA splicing events, and alternative translational and postranslational events. The polypeptide can be expressed in systems, e.g., cultured cells, which result in substantially the same postranslational modifications present when expressed the polypeptide is expressed in a native cell, or in systems which result in the alteration or omission of postranslational modifications, e.g., gylcosylation or cleavage, present when expressed in a native cell.

In one aspect, an SCP polypeptide has one or more of the following characteristics:

(i) it has the ability to inhibit cellular differentiation in to neuronal tissue;

(ii) it has a molecular weight, amino acid composition or other physical characteristic of SEQ ID NO:2, 4, or 6;

(iii) it has an overall sequence similarity of at least 50%, preferably at least 60%, more preferably at least 70, 80, 90, or 95%, with a polypeptide of SEQ ID NO:2, 4 or 6;

(iv) it can be found in non-neuronal cells;

(v) it can colocalize to the nucleus with REST/NRSF; and

(vi) it has the ability to dephosphorylate the CTD of RNA polymerase II.

In another embodiment the SCP protein, or fragment thereof, differs from the corresponding sequence in SEQ ID NO:2, 4, 6 or 8. In one embodiment it differs by at least one but by less than 50, 30, 15, 10 or 5 amino acid residues. In another aspect, it differs from the corresponding sequence in SEQ ID NO:2, 4, 6 or 8 by at least one residue but less than 20%, 15%, 10% or 5% of the residues in it (if this comparison requires alignment the sequences should be aligned for maximum homology.

In another embodiment, dominant negative mutant SCP polypeptides are provided. SEQ ID NO:10 and 12 are examples of SCP polypeptides that, when expressed in a cell, inhibit the activity of wild-type SCP.

Anti-SCP Antibodies

In another aspect, the invention provides an anti-SCP antibody. The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments which can be generated by treating the antibody with an enzyme such as pepsin.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length SCP protein or, antigenic peptide fragment of SCP can be used as an immunogen or can be used to identify anti-SCP antibodies made with other immunogens, e.g., cells, membrane preparations, and the like. The antigenic peptide of SCP should include at least 8 amino acid residues of the amino acid sequence shown in SEQ ID NO:2 and encompasses an epitope of SCP. Preferably, the antigenic peptide includes at least 10 amino acid residues, more preferably at least 15 amino acid residues, even more preferably at least 20 amino acid residues, and most preferably at least 30 amino acid residues.

Recombinant Expression Vectors, Host Cells and Genetically Engineered Cells

In another aspect, the invention includes, vectors, preferably expression vectors, containing a nucleic acid encoding an SCP polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a SCP nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., SCP proteins, mutant forms of SCP proteins, fusion proteins, and the like).

The recombinant expression vectors of the invention can be designed for expression of SCP proteins in prokaryotic or eukaryotic cells. For example, polypept ides of the invention can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

The invention further provides a recombinant expression vector comprising an SCP DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., Antisense RNA as a molecular tool for genetic analysis, Reviews—Trends in Genetics, Vol. 1(1) 1986.

A host cell can be any prokaryotic or eukaryotic cell. For example, an SCP protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., SCP DNA) into a host cell, including calcium phosphate or calcium chloride co-precipitation, DEAE-dextran-mediated transfection, lipofection, or electroporation ps Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to SCP proteins, have a stimulatory or inhibitory effect on, for example, SCP expression or SCP activity, or have a stimulatory or inhibitory effect on, for example, the expression or activity of a SCP substrate. Compounds thus identified can be used to modulate the activity of target gene products (e.g., SCP genes) in a therapeutic protocol, to elaborate the biological function of the target gene product, or to identify compounds that disrupt normal target gene interactions.

Compounds used in the methods described herein can be proteinaceous in nature, such as peptides (comprised of natural and non-natural amino acids) and peptide analogs (comprised of peptide and non-peptide components), or can be non-proteinaceous in nature, such as small organic molecules. The substance can also be a genetically engineered SCP protein with an altered amino acid sequence. These substances would be designed to bind to, or interact with the SCP protein based on the DNA or amino acid sequences of the SCP proteins described herein, or antibodies reactive with the SCP proteins described herein.

For example, a substance can be identified, or designed, that specifically interferes with the phosphatase activity of one, or more, SCP proteins thereby inhibiting RNA polymerase II holoenzyme activity. Monoclonal or polyclonal antibodies (e.g., the polyclonal antibodies described herein) specific for one, or more, of the SCP proteins can also be used to prevent, or inhibit, the SCP proteins from participating in the initiation of gene transcription.

In one embodiment, the invention provides assays for screening candidate or test compounds which are target molecules of a SCP protein or polypeptide or biologically active portion thereof. In another embodiment, the invention provides assays for screening candidate or test compounds which bind to or modulate the activity of a SCP protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

In one embodiment, an assay is a cell-based assay in which a cell which expresses a SCP protein or biologically active portion thereof is contacted with a test compound and the ability of the test compound to modulate SCP activity determined. Determining the ability of the test compound to modulate SCP activity can be accomplished by monitoring the bioactivity (i.e., phosphatase activity) of the SCP protein or biologically active portion thereof. The cell, for example, can be of mammalian origin.

Methods for Cell Differentiation

The invention encompasses methods for modulating or regulating the differentiation of a population of a specific progenitor cell into specific cell types comprising differentiating the progenitor cell under conditions suitable for differentiation and in the presence of one or more compounds of the invention. Alternatively, the stem or progenitor cell can be exposed to a compound of the invention and subsequently differentiated using suitable conditions. As used herein, compound includes small molecules, RNA (e.g. siRNA), DNA, chemical compositions and antibodies.

The invention also encompasses the modulation of stem or progenitor cells in vivo, in a patient to be treated. Thus, one or more of the SCP1, SCP2 or SCP3 inhibitory compounds of the invention, alone or in combination, may be administered to a patient. In various embodiments, such compounds may be administered concurrently or serially in combination with, for example, stem or progenitor cells, the differentiation of which has been modulated using one or more of the compounds of the invention; with treated stem or progenitor cells and untreated stem or progenitor cells. The compound and any treated or untreated cells may be administered together or separately; in the latter case, the cells or the compound(s) may be administered first.

In a specific embodiment, the present invention provides methods that employ SCP1, SCP2 and/or SCP3 inhibitors to modulate and regulate nerve tissue regeneration.

In other embodiments, the methods of the invention may be used to regulate the differentiation of e.g., a neuronal precursor cell or neuroblast into a specific neuronal cell type such as a sensory neuron (e.g., a retinal cell, an olfactory cell, a mechanosensory neuron, a chemosensory neuron, etc.), a motorneuron, a cortical neuron, or an interneuron. In other embodiments, the methods of the invention may be used to regulate the differentiation of cell types including, but not limited to, cholinergic neurons, dopaminergic neurons, GABA-ergic neurons, glial cells (including oligodendrocytes, which produce myelin), and ependymal cells (which line the brain's ventricular system). In yet other embodiments, the methods of the invention may be used to regulate the differentiation of cells that are constituent of organs, including, but not limited to, purkinje cells of the heart, biliary epithelium of the liver, beta-islet cells of the pancreas, renal cortical or medullary cells, and retinal photoreceptor.cells of the eye.

As used herein, the term “stem cell” refers to a master cell that can reproduce indefinitely to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells.

As used herein, “stem cell” includes embryonic and adult (somatic) stem cells. An adult stem cell is an undifferentiated cell found among differentiated cells in a tissue or organ, can renew itself, and can differentiate to yield the major specialized cell types of the tissue or organ. The primary roles of adult stem cells in a living organism are to maintain and repair the tissue in which they are found.

Any mammalian stem cell can be used in accordance with the methods of the invention, including but not limited to, stem cells isolated from cord blood (“CB” cells), placenta and other sources. The stem cells may include pluripotent cells, i.e., cells that have complete differentiation versatility, that are self-renewing, and can remain dormant or quiescent within tissue. The stem cells may also include multipotent cells or committed progenitor cells.

### EXAMPLES

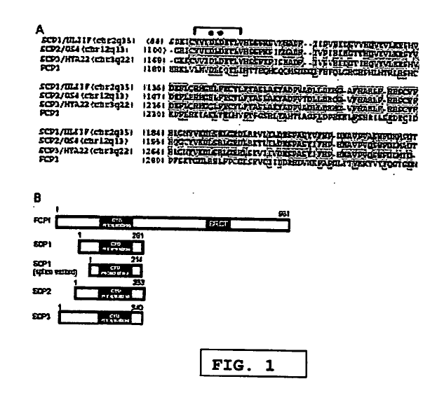

The alignment of three human proteins that are closely related to one another and have homology to the phosphatase domain of human FCP1 is shown in FIG. 1A. All contain the signature motif ΨΨΨDXDX(T/V)ΨΨ. The full-length 261 aa protein is encoded by 7 exons; a shorter NH₂ terminal splice version of 214 aa is present in EST databases. SCP1 has ˜20% homology to human FCP1 in the phosphatase domain while the 3 SCP proteins are >90% homologous in this region. SCP2/OS4 located on chromosome 12q13 was co-amplified with CDK4 in sarcomas (Su et al., (1997) Oncogene 15:1289-1294) and SCP3/HYA22 located on chromosome 3q22 was part of a large chromosome deletion in a lung carcinoma cell line. These represent a subset of proteins with putative. CTD phosphatase-like catalytic domains found in plants, yeast, nematodes and arthropods. The Drosophila and Anopheles genomes each contain a single highly conserved SCP ortholog. The SCP proteins lack the BRCT domain present in FCP1 (FIG. 1B).

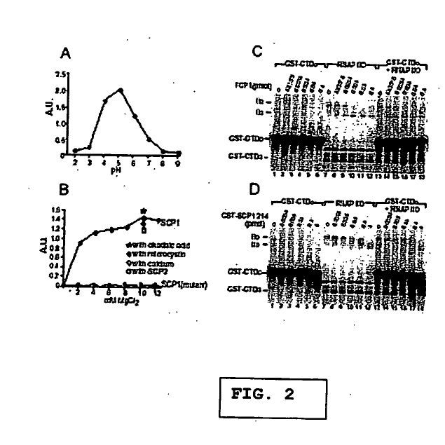

The SCP1 protein was expressed as a GST-fusion and both SCP1 261 and SCP1 214 were assayed using PNθP as substrate. The pH optimum for SCP1 phosphatase activity is near 5 (FIG. 2A). Phosphatase activity was Mg²⁺-dependent and resistant to the phosphatase inhibitors okadaic acid and microcystin (FIG. 2B). Ca²⁺ could not substitute for Mg²⁺. Mutations of Asp95 to Glu (D95E) had little to no effect on phosphatase activity whereas mutating Asp97 to Asn (D97N) in conjunction with the D95E mutation completely abolished phosphatase activity (FIG. 2B). SCP1 is thus a class 2C phosphatase whose activity is dependent on acidic residues in the conserved DXD motif. SCP2 exhibited similar phosphatase activity (FIG. 2B).

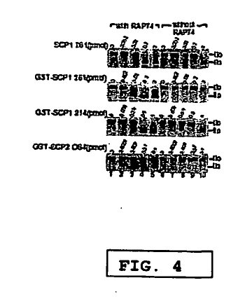

GST-CTDo and RNAP IIO were utilized as substrates and the activity of SCP1 was compared directly with that of FCP1. Recombinant CTDo (rCTDo) and RNAP IIO utilized as substrate in these experiments were prepared by the phosphorylation of purified GST-CTDa or RNAP IIA with casein kinase II (CKII) in the presence of [γ-³²P]ATP followed by phosphorylation with MAPK2/ERK2 in the presence of excess unlabeled ATP. MAPK2/ERK2 was used in these initial experiments because it phosphorylates both GST-CTDa and RNAP IIA with comparable efficiency. FCP1 converts RNAP IIO to RNAP IIA in a processive manner (FIG. 2C, lanes 7-12). Higher concentrations of FCP1 did not result in measurable dephosphorylation of rCTDo (FIG. 2C, lanes 1-6). GST-SCP1 214 catalyzed the dephosphorylation of both RNAP IIO and GST-CTDo with comparable efficiency (FIG. 2D). In contrast to FCP1, the SCP1 catalyzed dephosphorylation of RNAP IIO appears non-processive in that a number of phosphorylated intermediates are visible in SDS-PAGE. SCP1 is specific for dephosphorylation of the consensus repeat in that the phosphate at the CKII site is not removed. Mutant SCP1 (D95E, D97N) lacked activity on either substrate. SCP1 is a CTD phosphatase that acts on both RNAP IIO and rCTDo. SCP2 exhibits comparable CTD phosphatase activity when RNAP IIO is utilized as substrate (see e.g., FIG. 4).

SCP1 Preferentially Dephosphorylates Ser 5 of the CTD Heptad Repeat

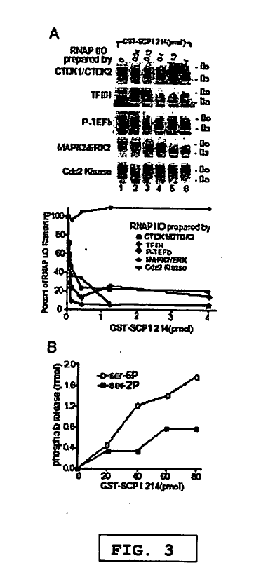

To determine the specificity of SCP1 with respect to its ability to dephosphorylate specific positions within the consensus repeat, RNAP IIO isozymes were prepared in vitro by the phosphorylation of RNAP IIA with CTD kinases of known specificity. TFIIH, P-TEFb and MAPK2/ERK2 preferentially phosphorylate Ser 5 when synthetic peptides serve as substrate whereas Cdc2 kinase phosphorylates Ser 2 and Ser 5. Although the specificity appears relaxed when RNAP II serves as substrate, RNAP IIO prepared with Cdc2 kinase is clearly distinct from RNAP IIO generated by other CTD kinases. Results presented in FIG. 3A indicate that RNAP IIO, prepared by the phosphorylation of RNAP IIA with distinct CTD kinases, exhibit a differential sensitivity to dephosphorylation with SCP1. SCP1 most efficiently dephosphorylates RNAP IIO generated by TFIIH and was unable to dephosphorylate RNAP IIO prepared with Cdc2 kinase. SCP1 was also unable to dephosphorylate RNAP IIO generated by Abl tyrosine kinase. The dephosphorylation of RNAP IIO isozymes prepared with P-TEFb, MAPK2/ERK2 and CTDK1/CTDK2 occurred at a reduced rate relative to that of RNAP IIO prepared with TFIIH. Furthermore, while the dephosphorylation reaction appears processive for RNAP IIO prepared by TFIIH, it is clearly non-processive for RNAP IIO generated by MAPK2/ERK2. In contrast FCP1 shows no preference for RNAP IIO generated by TFIIH and efficiently dephosphorylates RNAP IIO generated by Cdc2 kinase. These results indicate that SCP1 differs from FCP1 in substrate specificity, showing relative preference for the dephosphorylation of Ser 5 in the heptad repeat.

A synthetic 28 aa peptide containing 4 heptad repeats phosphorylated exclusively on Ser 2 or on Ser 5 was dephosphorylated in the presence of increasing amounts of SCP1. As shown in FIG. 3B, SCP1 preferentially dephosphorylates the Ser 5 phosphopeptide compared to the Ser 2 phosphopeptide. This substrate specificity contrasts to that reported for FCP1 from S. pombe which referentially dephosphorylate the Ser 2 phosphopeptide. Mammalian FCP1, within a comparable concentration range, did not act on either phosphopeptide. These results using synthetic phosphopeptide substrates confirm that SCP1 preferentially dephosphorylates Ser 5 phosphate of the CTD.

Effect of RAP74 on the Activity of SCP1

The RAP74 subunit of TFIIF stimulates CTD phosphatase activity of FCP1. Furthermore, the domains of FCPL that bind RAP74 are required for FCP1-dependent viability in S. cerevisiae. Therefore, it was of interest to determine if RAP74 can also influence the activity of SCP. CTD phosphatase activity was measured at low enzyme concentrations to more readily detect stimulatory effects of RAP74. As shown in FIG. 4, RAP74 shifted the dose response curve for SCP1 catalyzed dephosphorylation of RNAP IIO to an approximately 10-fold lower concentration. The CTD phosphatase activity of the GST-fusion forms of SCP1 261, SCP1 214 and SCP2 were also enhanced by RAP74. In support of the conclusion that RAP74 stimulates the activity of SCPs, RAP74 bound directly to GST-SCP1 but not to GST. The binding and stimulatory effects of RAP74 suggest that TFIIF is important for optimal CTD phosphatase activity for both FCP1 and SCP1.

SCP1 Nuclear Localization

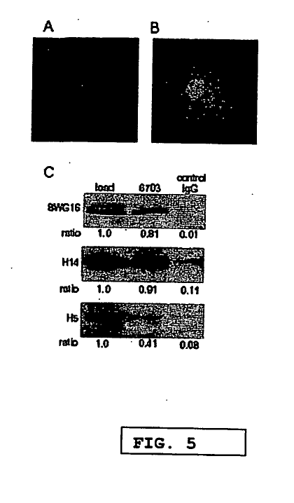

Although SCP1 lacks an obvious nuclear localization sequence, it is found in the nucleus. Immunofluorescence microscopy using a rabbit polyclonal anti-SCP1 antibody demonstrated nuclear localization of endogenous SCP1 in COS7 cells (FIG. 5B). Co-staining with DAPI for nuclear identification and with the early endosomal marker EEa1 for cellular detail confirmed the specific localization of SCP1 in nuclei (FIG. 5 panels A and B).

Co-immunoprecipitation was used to assess the association of SCP1 with RNAP II. Sepharose-immobilized anti-SCP1 IgG 6703 was used to immunoisolate SCP1 from COS7 cells. Immunoisolates were resolved by SDS-PAGE and blotted with anti-RNAP II antibodies. As shown in FIG. 5C, RNAP II was present in SCP1 immunoprecipitates indicating that SCP1 and RNAP II either interact directly or are in the same macromolecular complex. To determine whether SCP1 preferentially associated with either Ser 2 or Ser 5 phosphorylated RNAP IIO, lysates were prepared in the presence of EDTA, to inhibit phosphatase activity. SCP1 immunoprecipitates were then blotted with monoclonal antibodies specific for Ser 2 phosphate (H5) and Ser 5 phosphate (H14). Both forms of RNAP IIO were present in COS7 cell lysates. Ser 5 phosphate-enriched RNAP IIO appeared to be preferentially associated with SCP1 in immunoprecipitates as indicated by the ratios of co-immunoprecipitated RNAP IIO relative to the amount of RNAP IIO contained in the extract (FIG. 5C).

SCP1 Affects RNAP II Transcription in Vivo

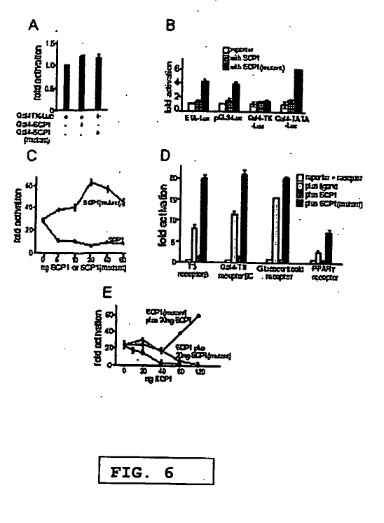

To assess the effect of SCP1 on transcription in vivo, the activity of a variety of luciferase reporter gene constructs was examined in the presence or absence of cotransfected SCP1. Targeting a Gal 4-DNA binding domain SCP1 fusion or the phosphatase-inactive Gal 4-SCP1 mutant upstream of a thymidine kinase promoter-luciferase reporter (Gal 4-TK-Luc) had no significant effect on transcriptional activity (FIG. 6A). Untethered SCP1 in the presence of several reporter constructs had no significant effect on reporter gene expression whereas the inactive mutant resulted in a significant stimulation of expression from the ElALuc, pGL3-Luc and Gal4-TATA-Luc constructs (FIG. 6B). The phosphatase-minus SCP1 mutant increased luciferase activity 1.5- to 6-fold.

In contrast, WT or phosphatase-inactive SCP1 affected reporter gene expression from a variety of regulated promoters. Luciferase activity from a Gal 4-TK-Luc reporter that was strongly stimulated by co-expressing a Gal 4-VP16 fusion protein (30-fold stimulation) was strongly inhibited by SCP1 (FIG. 6C). In contrast, phosphatase-inactive SCP1 enhanced Gal 4-Vpl6-stimulated activity about 2-fold.

Opposing effects of active SCP1 and the inactive mutant SCP1 were observed using a number of inducible promoter-reporter constructs. Ligand-activated T₃ receptor activity on a DR+4 TRE-TK-Luc reporter gene was inhibited by active SCP1 and enhanced by inactive SCP1 (FIG. 6D). Similar results were obtained when the C-terminus of T₃Rβ was fused to the Gal 4-DNA binding domain (Gal 4-T₃RβC) and targeted to Gal 4-TK-Luc. SCP1 also inhibited dexamethasone-stimulated glucocorticoid receptor activity on a GRE-TK-Luc construct whereas mutant SCP1 significantly enhanced activity (FIG. 6D). Finally, SCP1 inhibited ligand-activated PPARγ receptor activity assayed on a PPARγ promoter response element and mutant SCP1 enhanced activity (FIG. 6D). A similar response pattern was observed when the Gal 4-DNA binding domain was fused to the C-terminus of PPARγ and targeted to Gal 4-TK-Luc. The same pattern of responses was observed in HEK293, COS-7 and CV-1 cells.

The competing effects of SCP1 and mutant SCP1 were further examined using a rat insulin promoter-luciferase construct. There is strong synergy on this promoter between the bHLH protein E47 and the LIM-homeodomain protein LMX1 which bind to adjacent DNA target sites (39,40). Co-expression of LMX1-and E47 enhanced luciferase activity 25-fold from the rat insulin 1 promoter (FIG. 6E). When phbsphatase-inactive SCP1 was held constant, increasing amounts of SCP1 inhibited luciferase expression (FIG. 6E). In the presence of mutant SCP1, the SCP1 inhibition curve was right-shifted (˜20% inhibition with 40 ng SCP1 plasmid plus 20 ng mutant SCP1 plasmid vs. >90% inhibition with 40 ng SCP1 plasmid alone (FIG. 6E)). With a constant input of SCP1 plasmid, increasing amounts of mutant SCP1 not only blocked the inhibitory effects of SCP1, but enhanced activity significantly. The stimulatory effects of transfected phosphatase-inactive SCP1 are thus consistent with it acting as a dominant negative inhibitor of wild-type SCP activity.

Acquisition of the CTD of RNAP II allows extensive protein interactions and is thought to have been an important step in the evolution of complex patterns of regulated gene expression. Most importantly, the CTD can exist in multiple conformations thereby facilitating the recruitment of different multiprotein complexes at specific points in the transcription cycle. Both the site of phosphorylation within the consensus repeat, Ser 2 or Ser 5, and the extent of phosphorylation of the CTD control many aspects of RNAP II transcription, including the recruitment of RNAP II to the preinitiation complex, initiation, capping, elongation, splicing and polyadenylation. Ser 2 and Ser 5 within the consensus CTD repeat are essential residues and distinct CTD.kinases catalyze phosphorylation at these sites (West and Corden, (1995) Genetics 140:1223-1233). To date, a single CTD phosphatase, FCP1, has been implicated in removing phosphates (for review see Lin et al., (2002) Prog. Nucl. Acid Res. Mol. Biol. 72:333-365).

SCP1 preferentially catalyzes dephosphorylation of RNAP IIO phosphorylated by TFIIH. RNAP IIO phosphorylated by P-TEFb and MAPK2/ERK2 are also dephosphorylated by SCP1 but at a reduced rate. RNAP IIO phosphorylated by Cdc2 kinase, which preferentially phosphorylates Ser 2 with some phosphorylation at Ser 5, is not a substrate for SCP1. The preferential dephosphorylation of Ser 5 by SCP1 was confirmed using a 4 heptad repeat peptide substrate. Interestingly, the specificity of SCP1 contrasts with that of S. pombe FCP1 which prefers Ser 2 with a similar peptide substrate (Hausmann and Shuman, (2002) J. Biol. Chem. 277:21213-21220). However, FCP1 dephosphorylates Ser 2 phosphates and Ser 5 phosphates with comparable efficiency when native RNAP IIO serves as substrate in vitro (Lin et al., (2002) J. Biol. Chem. 277:45949-45956). The relative specificity of FCP1 also differs from SCP1 in that unlike FCP1, SCP1 shows preference for the dephosphorylation of TFIIH phosphorylated RNAP IIO.

It is clear from the results presented in FIG. 2C and FIG. 2D that, when RNAP IIO phosphorylated with MAPK2/ERK2 is utilized as substrate, the specific activity of SCP1 is substantially lower than that of FCP1. This difference in specific activity is in part due to the fact that MAPK2/ERK2 phosphorylated RNAP IIO is an especially poor substrate for SCP1 (FIG. 3) whereas FCP1 dephosphorylates different isozymes of RNAP IIO with comparable efficiency. The amount of SCP1 required to dephosphorylate MAPK2/ERK2 phosphorylated RNAP IIO is 50 to loo fold higher than that required to dephosphorylate TFIIH phosphorylated RNAP IIO. Furthermore, the BRCT domain in FCP1 that facilitates its interaction with RNAP II is absent from SCP1. The finding that SCP1 dephosphorylates rCTDo and RNAP IIO with comparable efficiency whereas FCP1 does not dephosphorylate rCTDo even at concentrations 100 times higher than that required to dephosphorylate RNAP IIO, indicate that the interaction of SCP1 and FCP1 with the CTD require different molecular interactions.

During the transcription cycle, protein complexes assemble and disassemble on the CTD in a dynamic and regulated manner. Ser 5 phosphorylation is detected primarily at the promoter region, whereas Ser 2 phosphorylation is seen in coding regions. Phosphorylation of Ser 5 facilitates recruitment of the capping enzymes and allosterically activates their activity (Pei et al., (2001) J. Biol. Chem. 276:28075-28082). Given the preference of SCP1 for phosphoserine 5, SCPs are candidates for acting early in the transcription cycle.

Like FCP1, SCP1 phosphatase activity is found in a complex with RNAP II and its activity is stimulated by RAP74. Mapping studies indicate that the C-terminal domain of FCP1 distal to the phosphatase domain interacts with TFIIF. A region near the C-terminus of SCP1 shares homology with the putative RAP74 interaction domain in CP1.

The results of reporter gene assays indicate that over-expression of either WT or mutant SCP1 can influence gene expression. The overexpression of mutant SCP1 activates transcription several fold from nearly all promoters examined (FIG. 6B, C and D) whereas overexpression of WT SCP1 appears to selectively inhibit activated transcription from a variety of inducible promoter-reporter gene constructs (FIG. 6D). Because mutant SCP1 is competitive with SCP1, the stimulatory effects are consistent with partial inhibition of endogenous SCP.

Extinction of SCP Expression in Differentiating Nervous Tissue

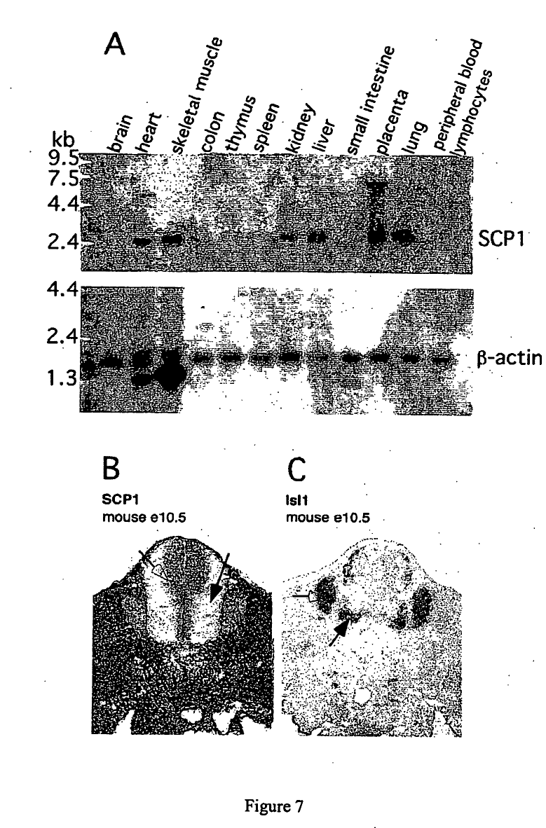

Northern analysis revealed that SCP1 is widely expressed with the highest levels observed in skeletal muscle and low to absent expression in brain (FIG. 7A). To determine whether a specific SCP family member was expressed in brain, probes specific for SCP 2 and SCP3 were also used. SCP2 and SCP3 expression was also very low in brain relative to other tissues indicating that expression of the entire SCP family is largely excluded from nervous tissue. These results confirm low expression of SCP1 and SCP2 in brain.

To examine the pattern of expression of SCPs in the developing nervous system, in situ hybridization was carried out in mice at e 10.5. SCP1 is widely expressed in cells surrounding the developing spinal cord and is expressed in proliferating neuroepithelium adjacent to the neural tube (FIG. 7B). However, SP1 is absent from the differentiating spinal cord lateral to the proliferating zone where neuronal differentiation markers are expressed (FIG. 7C). Similarly SCP1 is expressed in proliferating neuro-epithelium adjacent to the 3rd ventricle but is absent from surrounding differentiated neuronal cells. This pattern of expression parallels that of REST/NRSF whose expression is widespread in non-neuronal tissue but excluded from neuronal tissues.

SCP1 is Found in a Complex with REST/NRSF at RE-1 DNA Elements

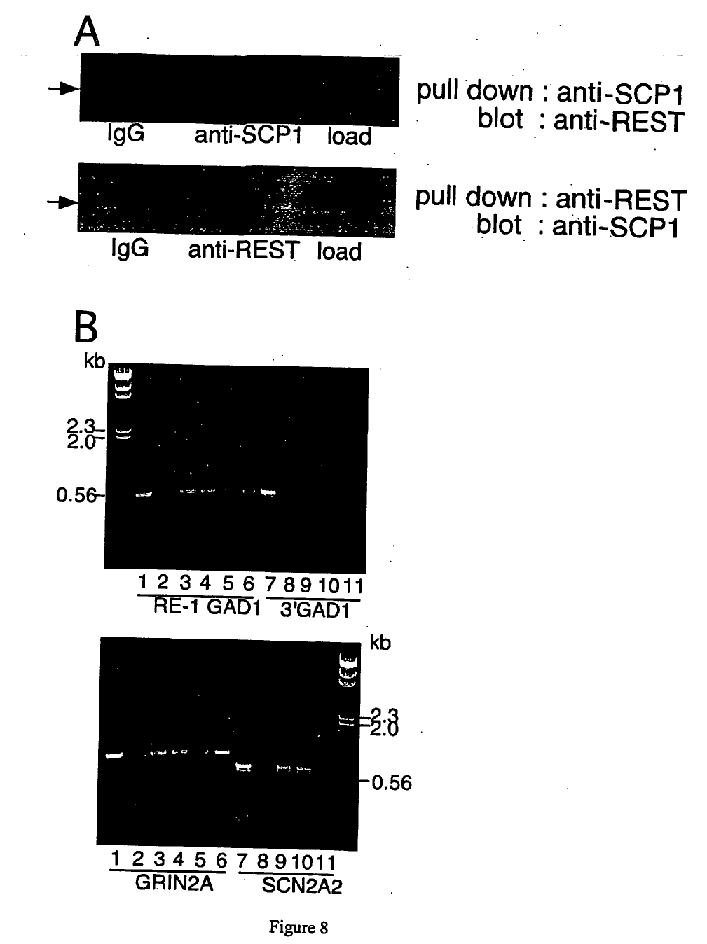

SCP1 co-immunoprecipitates with RNAP II suggesting it is part of a molecular complex with a subset of RNAP II molecules. The pattern of exclusion of SCPs from differentiated nervous tissue indicates that these phosphatases function with REST/NRSF to silence neuronal gene expression in non-neuronal tissues. The interactions between SCP1 and REST/NRSF were determined using co-immunoprecipitation. REST/NRSF immunoprecipitates contained SCP1; conversely SCP1 immunoprecipitates contained REST/NRSF (FIG. 8A) indicating that the two proteins exist in a molecular complex in non-neuronal cells.

Chromatin immunoprecipitation (ChIP) with anti-SCP antibodies and PCR primers specific for the REST/NRSF binding elements of the Na+ channel II (SCN2A2), glutamate receptor (GRIN2A) and glutamic acid decarboxylase (GAD1) genes was used to determine that SCP1 was part of the REST/NRSF complex. As shown in FIG. 8B, SCP is specifically associated with the REST binding sites of the SCN2A2, GRIN2A and GAD1 genes as confirmed by parallel immunoprecipitations using anti-REST/NRSF antibodies but not by control immunoglobulin. Anti-REST/NRSF and anti-HP1 antibodies similarly indicated these proteins did not localize to the region of the 3′ region of the GAD1 gene. Using antibodies to other components of the REST complex, ChIP analysis indicated localization of HDAC1, HP1 (hetero chromatin protein 1) and MeCp1 at the RE-1 element (FIG. 8B). These proteins along with CO-REST are part of the REST/NRSF complex. These results indicate that SCP is a component of the REST/NRSF complex located at the RE-1 DNA binding elements of neuronal genes.

SCP1 Expression in Embryonic Stem Cells

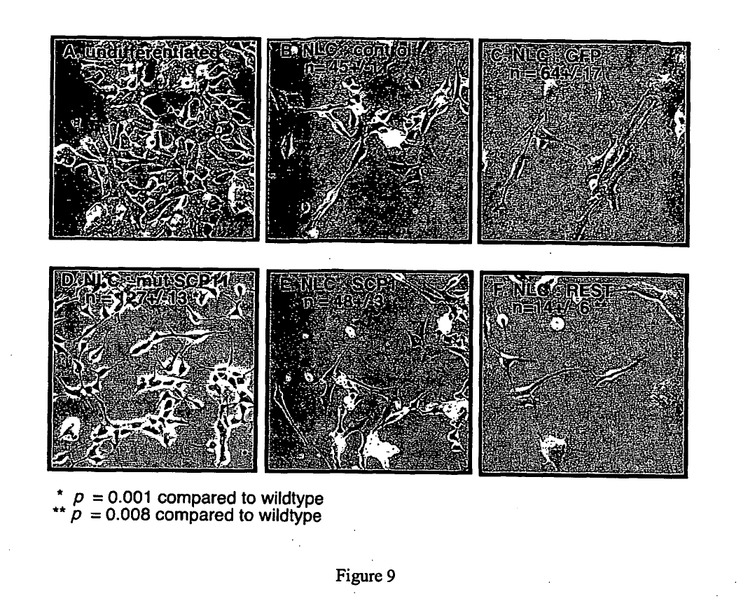

P19 mouse embryonic stem cells can be induced to undergo neuronal differentiation by treatment with retinoic acid under defined cell culture conditions. Clonal P19 cell lines expressing SCP1, a mutant phosphatase-inactive D96E/D98N SCP1 that acts as a dominant negative, REST/NRSF or GFP as a control, were generated to determine the pattern of expression and effects of SCP1 on neuronal differentiation. Under defined conditions >90% of viable P19 cells undergo morphological differentiation into neurons (FIG. 9A, FIG. 9C). Expression of REST/NRSF, which did not affect stem cell proliferation, prevented neuronal differentiation and most cells died under selective neuronal induction conditions (FIG. 9F). Consistent with a requirement for REST/NRSF, whose expression is extinguished upon neuronal differentiation, SCP1 did not affect the extent of neuronal differentiation (FIG. 9D). However dominant negative SCP1 increased the extent of precursor differentiation into neurons >2-fold (FIG. 9E).

SCP1 and REST/NRSF are expressed in replicating P19 stem cells but the expression of both genes is extinguished upon differentiation into neurons. concomitantly cells acquire expression of the neuron-specific gene β-tubulin. These results indicate that both SCP and REST/NRSF expression are extinguished upon neuronal differentiation. Moreover blocking SCP effects in P19 stem cells by dominant negative SCP increased the fraction of stem cells that differentiated into neurons.

Suppression of SCP Enhances Neuronal Gene Expression

Although a Drosophila REST/NRSF neuronal gene silencing mechanism orthologous to that in seen mammals has not been defined, Drosophila also suppress neuronal gene expression in non-neuronal cells. Because examination of the fly databases revealed no. P element insertions or specific deletions in the SCP locus, silencing RNA (siRNA dSCP) was used to “knock down” Drosophila SCP (dSCP) expression. The Drosophila genome contains a single SCP ortholog with high homology to human SCP1 (75% amino acid identity), making use of siRNA more feasible than in mammalian cells, which express 3 SCPs. Because the level of dSCP expression is relatively stable from the earliest times of analysis of Drosophila embryos there is likely a strong maternal component of dSCP mRNA during early development. The Drosophila gene product expressed as a GST-fusion protein in bacteria exhibited phosphatase activity similar to that described for human SCPs.

S2 cells, which were initially established from 20-22 hr., Drosophila embryos were treated with a 700 bp siRNA dSCP to decrease dSCP expression and effects on expression of a variety of neuronal and non-neuronal genes was achieved without obvious effects on S2 cell growth or morphology. dSCP mRNA was reproducibly decreased >80% by 24 hr. Decreasing dSCP had no effect on expression of glyceraldehyde phosphate dehydrogenase (GAPDH), ribosomal protein S35 or β-actin mRNAs. In contrast siRNA dSCP enhanced expression of a set of neuronal genes: the sodium channel II gene (NaChII), the glutamate receptor, ELAV, β-tubulin and glial cell missing (GCM) 2 to >10 fold. The mammalian orthologs of NaChII, glutamate receptor and b-tubulin are classical neuronal genes that contain RE-1 elements. Some gene transcripts could not be detected: synapsin, stathmin, choline acetyltransferase (ChoAcTR), neurofilament and a non-neuronal oxygenase. Myosin light chain kinase, which was robustly expressed in control S2 cells, was increased 2.5-fold when dSCP was suppressed. In S2 cells expression of a set of neuronal genes is thus markedly enhanced when dSCP is decreased suggesting dSCP acts to suppress their transcription. The effects of siRNA dSCP to increase expression of the glial specific gene ELAV indicates that dSCP acts to repress both neuronal and glial-specific gene expression. This is consistent with lack of expression of SCP in mammalian brain and spinal cord that contain both glial and neuronal elements.

FIG. 1A shows the sequence alignment and relationship of 3 small phosphatases with FCP1. Bracket indicates the conserved signature motif and * indicate critical Asp residues involved in phosphatase activity. Previous descriptive names and chromosome locations are indicated. Multiple alignments were done using Clustal W algorithm with vector NTI Suite (Informax). FIG. 1B provides diagrams of the domain structures of FCP1 and SCP proteins.

FIG. 2A is an autoradiogram showing the pH optimum for SCP1 utilizing synthetic peptide substrates. GST-SCP1 214 (40 pmol) was incubated with 20 mM PNθP for 60 min at 30° C. and phosphatase activity measured by the change in A₄₁₀. FIG. 2B provides data showing the divalent metal ion requirement for SCP1 activity. The phosphatase activity of GST-SCP1 214 (40 pmol) was measured in the presence of 20 mM PNθP and varying concentrations of [Mg²⁺] or [Ca²⁺]. Activity was also measured in the presence of 1-10 μM okadaic acid and 1-10 μM microcystin. The 10 μM concentration is shown. Mutant SCP1 (D96E D98N) was inactive ( - - - ). SCP2 also exhibited phosphatase activity. (□).

FIGS. 2C and 2D show a CTD phosphatase assay of FCP1 and SCP1 on GST-CTDo and RNAP IIO prepared by MAPK2/ERK2. Increasing amounts of FCP1 or GST-SCP1 214 were assayed in the presence of 75 fmol GST-CTDo (lanes 1-6), 75 fmol RNAP IIO (lanes 7-12), or 75 fmol GST-CTDo and 75. fmol RNAP IIO (lanes 13-18). Both GST-CTDo and RNAP IIO substrates were prepared by the in vitro phosphorylation of CKII-labeled GST-CTDa or RNAP IIA by MAPK2/ERK2. All reactions were carried out in the presence of 7 pmol RAP74. CTD dephosphorylation of both GST-CTDo and RNAP IIO is shown by the increase in mobility of GST-CTDo to GST-CTDa and subunit IIo to IIa, respectively. The difference in the intensity of radiolabeled GST-CTDoand that of radiolabeled subunit IIo is not a reflection of a difference in the amount of substrates present, but of the higher efficiency with which CKII incorporates radiolabeled phosphates onto the most C-terminal serine of GST-CTDa compared to subunit. IIa.

FIG. 3A shows dephosphorylation of RNAP IIO prepared with various CTD kinases. Increasing amounts of GST-SCP1 214 were assayed in the presence of 3.7 fmol RNAP IIO prepared by CTDK1/CTDK2, TFIIH, P-TEFb, MAPK2/ERK2 and Cdc2 kinase. All reactions were carried out in-the presence of 7 pmol. RAP74. CTD dephosphorylation of RNAP IIO isozymes. by qSTSCP1 214 is shown by an increase in mobility of subunit IIo to that of IIa. The results are summarized in the graph showing the percent of RNAP IIO remaining as a function of increasing SCP1 concentrations.

FIG. 3B shows the effects of GST-SCP1 214 on a 28 aa peptide consisting of heptad repeats containing either Ser 5 phosphate or Ser 2 phosphate. The indicated amounts of. SCP1 were incubated with the phosphopeptide substrate and phosphate released was measured as described infra.

FIG. 4 shows the effect of RAP74 on CTD phosphatase activity of SCP1 and SCP2. Increasing amounts of the indicated forms of SCP1 and SCP2 were assayed in the presence of 14.4 fmol RNAP. IIO prepared by TFIIH. Reactions were carried out in the presence and in the absence of 7 pmol RAP74.

FIGS. 5A and 5B show cells co-stained for the endosomal marker EEA1 using mouse anti-EEa1 and Alexa Fluor 594 conjugated goat anti-mouse (red). Nuclei were detected with DAPI (blue) (5A). Immunofluorescence microscopy detection of endogenous SCP1 using rabbit polyclonal IgG 6307 and Alexa Fluor 488 conjugated goat anti-rabbit IgG (green) (5B). Second antibodies alone served as a control in all cases.

FIG. 5C shows co-immunoprecipitation of RNAP II and endogenous SCP1. Extracts from untransfected COS7 cells were immunoprecipitated using sepharose-immobilized anti-SCP1 IgG 6703 or sepharose-immobilized control IgG. Immunoprecipitates were resolved on SDS-PAGE and blotted using anti-RNAP II antibody (8WG16) and with the Ser 2 phosphate epitope specific antibody H5 and the Ser 5 phosphate epitope specific antibody H14. RNAP IIO present in COS7 lysates is shown on the left (5% load) and the relative ratio of each form of RNAP II in SCP1 immunoprecipitates to that in lysates is given.

FIG. 6A shows the effect of targeted SCP1 261 on reporter gene expression. Gal4-DNA binding domain-SCP1 fusion protein expression plasmids-were cotransfected in HEK293 cells with a Gal 4-TKLuc reporter plasmid and luciferase gene expression quantitated. In all panels, results of triplicate transfections are shown +/− SD. Data is expressed as fold-activation experimental/control). FIG. 6B shows the effect of SCP1 261 and mutant SCP1 261 on basal promoter activity. The indicated reporter plasmids were cotransfected with SCP1 or phosphatase-inactive SCP1. FIG. 6C shows differential effects of SdP1 261 and phosphatase-inactive SCP1 261 on Gal 4-VP16 stimulated gene expression. The indicated amounts of SCP1 or mutant SCP1 expression plasmids were cotransfected in HEK293 cells along with Gal 4-VP16 expression and Gal 4-TK-Luc reporter plasmids and luciferase activity was quantitated. FIG. 6D shows the effect of SCP1 261 and phosphatase-inactive SCP1 261 on ligand activated receptor activity. The indicated receptor expression plasmids were cotransfected with their cognate promoter-reporter plasmids with or without SCP1 or mutant SCP1 expression plasmids. Cells were treated or untreated with receptor-specific ligands as described infra. FIG. 6E shows the competitive effects of mutant SCP1 261 with SCP1 261. The indicated concentrations of SCP1 and mutant SCP1 expression plasmids were cotransfected with LMX1 and E47 expression plasmids and the rat insulin 1 promoter-luciferase reporter gene. Luciferase activity was measured as described.

FIG. 7A shows Northern blot analysis of the expression of SCP1 in human tissues. FIG. 7B shows in situ hybridization analysis of expression of SCP1 in e 10.5 mouse cervical spinal cord. SCP1 is widely expressed in cells surrounding the developing spinal cord and in proliferating neuroepithelium adjacent to the neural tube (open arrow). SCP1 expression is absent from the differentiating spinal cord lateral to the proliferation zone (closed arrow). FIG. 7C provides in situ analysis of the expression of isl-1. Isl-1 is expressed in ventral motor neurons (closed arrow) and dorsal sensory neurons (open arrow), areas of the developing spinal cord where SCP1 is not expressed.

FIG. 8A provides data indicating that SCP1 and REST/NRSF co-immunoprecipitate. HEK 293 cell extracts were immunoprecipitated using anti-SCP (upper panel) or anti-REST/NRSF (lower panel) antibodies, immunoprecipitates were resolved on SDS PAGE and associated proteins were identified by Western blotting using anti-REST/NRSF and anti-SCP1 antibodies. FIG. 8B shows chromatin immunoprecipitation using anti-SCP antibody. ChIP assays of HEK 293 cells using 6703 anti-SCP, anti-REST/NRSF, anti HDAC1, anti-HP1 or control IgG. PCR primers (see Table 1) specific for the RE-1 elements of GAD1, GRINA 2A, SCN2A2 genes and for the 3′ intron-exon region of GAD1 were used for RT-PCR. Upper panel: lanes 1-6=RE1 element of the GAD 1 gene; lanes 7-11=3′ region of GAD1 gene; lanes 1 and 7=load (1%); lanes 2 and 8; control IgG; lanes 3 and 9, anti-SCP; lanes 4 and 10, anti-REST/NRSF.; lanes 5 and 11 anti-HDAC1; lane 6, anti-HP1. Lower panel: lanes 1-6=RE1 element of GRINA2 gene; lanes 7-11 RE1 element of SCN2A gene; lanes 1 and 7 =load (1%); lanes 2 and 8, control IgG; lanes 3 and 9, anti-SCP; lanes 4 and 10, anti-REST/NRSF; lane 6, anti-HP1; lane 11, anti-HDAC1.

FIG. 9A through 9F shows SCP and REST/NRSF effects on neuronal differentiation of P19 cells. WT P19 cells and clonal lines expressing GFP (vector control), SCP1, mutant phosphatase-inactive SCP1 or REST/NRSF were induced to differentiate into neuron like cells (NLC) by treatment with retinoic acid and growth in selective medium a, undifferentiated; B differentiated P19 cells. P19 cells, C, differentiated GFP expressing P19 cells; D, differentiated SCP1-expressing P19 cells; F, differentiated REST/NRSF-expressing P19 cells. Numbers of NLC per field are shown. *p=0.001 compared to WT 19 cells; **p=0.008 compared to WT P19 cell using student's t-test.

In addition, the inventors have found that silencing of SCP enhances expression of neuronal and glial genes. FIG. 10 shows the quantitation of transcripts using real time quantitative RT-PCR. S2 cells were either untreated (−) or treated (+) with siRNA dSCP for 24 hr. Total RNA was prepared, DNAse treated and primer pairs specific for the coding sequence of each gene were used for qPCR using SYbR green chemistry.

### Materials

SCP1 and SCP2 were obtained as EST clones from Resgen. The full-length cDNA for SCP1 (261 aa, accession BE300370), the cDNA encoding the spliced variant of SCP1 (214 aa, accession L520011) and SCP2 (accession AL520463) were subcloned into EcoR1-Xho1 sites of pGEX4T-1 and pcDNA3Flag vectors by PCR. The D96E, D98N mutant of SCP1 261 (nucleic acid SEQ ID NO:9 encoding amino acid SEQ ID NO:10) and the corresponding mutant of SCP1 214, D48E and D50N (nucleic acid SEQ ID NO:11 encoding amino acid SEQ ID NO:12), were generated by QuikChange (Stratagene). The amino acid sequence of SEQ ID NO:10 is identical to that of SEQ ID NO:2 except that the aspartic acid (D) at position 96 in SEQ ID NO:2 has been changed to glutamic acid (E) in SEQ ID NO:10 and the aspartic acid (D) at position 98 of SEQ ID NO:2 has been changed to asparagine (N) in SEQ ID NO:10. In addition, the amino acid sequence of SEQ ID NO:12 is identical to that of SEQ ID NO:8 except that the aspartic acid (D) at position 48 in SEQ ID NO:8 has been changed to glutamic acid (E) in SEQ ID NO:12 and the aspartic acid (D) at position 50 of SEQ ID NO:8 has been changed to asparagine (N) in SEQ ID NO:12. GST fusions were purified by glutathione-sepharose chromatography and SCP1 261 was generated by cleavage at the thrombin site. encoded in the vector. Recombinant FCP1 was expressed and purified as described previously (23).

Human recombinant casein kinase II (CKII) and mouse recombinant MAPK2/ERK2 were obtained from Upstate Biotechnology. Human CTDK1/CTDK2 were purified as described by Payne and Dahmus (28). Human TFIIH was obtained as described (29). Human P-TEFb was partially purified from HeLa S-100 extract by chromatography on Heparin-Sepharose (Amersham Biosciences), DEAE 15HR (Millipore) HiTrap S and Phenyl-Superose (both from Amersham Biosciences). P-TEFb was dialyzed against 25 mM Hepes, pH 7.9, 20% glycerol, 25 mM KCl, 0.1 mM EDTA, 1 mM DTT, 1 mM PMSF. Human recombinant Cdc2 kinase was purchased from New England Biolabs. Rabbit anti-SCP1 IgG was prepared by ammonium sulfate fractionation and protein G-sepharose chromatography. RNAP II antibodies (8WG16, H5 and H14) were obtained from Covance.

Preparation and Purification of ³²P-RNAP IIO Isozymess and ³²P-GST-CTDo: Calf thymus RNAP IIA was purified by the method of Hodo and Blatti (30) with modifications as described by Kang and Dahmus (31). Specific isozymes of ³²p-labeled RNAP IIO were prepared by phosphorylation at the most C-terminal serine (CKII site) in the largest subunit of purified RNAP IIA with recombinant CKII and [K-³²P]ATP, followed by CTD phosphorylation in the presence of 2 mM ATP with either purified CTDK1/CTDK2, TFIIH, P-TEFb, recombinant MAPK2/ERK2 or recombinant Cdc2 kinase. The RNAP IIO isozymes were individually purified over a DE53 column with a step elution of 500 mM KCl (28). Because only the most C-terminal serine is labeled with ³²p and lies outside. the consensus repeat, dephosphorylation by CTD phosphatase results in an electrophoretic mobility shift in SDS-PAGE of subunit IIo to the position of subunit IIa without the loss of label. Similarly, ³²P-labeled GST-CTDo was prepared from GST-CTDa by CKII followed by MAPK2/ERK2. GST-CTDo was purified over a glutathione-agarose column with a step elution of 15 mM glutathione.

Phosphatase Assays: PNθP reaction mixtures (200 μl) containing 50 mM Tris-acetate, pH 5.5, 10 mM MgCl2, 0.5 mM DTT, 10% glycerol, 20 mM PNθP and recombinant proteins were incubated for at 30° C. for 1 hr. The reactions were quenched by adding 800 μl of 0.25 N NaOH. Release of pNθ was determined by measuring A410.

N-terminal biotinylated CTD phosphopeptides, comprised of 4 tandem repeats YSPTSPS and containing phosphoserine at position 2 or position 5, were synthesized (Alpha Diagnostics, San Antonio, Tex.). Phosphatase reaction mixtures (50 μl) containing 50 mM Tris-acetate, pH 5.5, 10 mM MgCl2, 0.5 mM DTT, 10% glycerol, 25 μM of phosphopeptide and wild type or mutant SCP1 were incubated for 60 mins at 37° C. The reactions were quenched by adding 0.5 ml of malachite green (Biomol). Phosphate release was measured at A₆₂₀ and quantified relative to a phosphate standard curve.

CTD phosphatase assays utilizing RNAP IIO and GST-CTDo as substrate were performed as described previously (32) with minor modifications. Reactions were performed in 20 μl of CTD phosphatase buffer (50 mM Tris-HCl, pH 7.9, 10 mM MgCl2, 20% glycerol, 0.025% Tween 80, 0.1 mM EDTA, 5 mM DTT) in the presence of 20 mM KCl. Each reaction contained specified amounts of GST-CTDo and/or RNAP IIO and was carried out in the presence of 7 pmol RAP74. Reactions were initiated by the addition of FCP1 or SCP1 and incubated at 30° C. for 30 minutes. Assays were terminated by the addition of 5× Laemmli buffer, and RNAP II subunits and GST-CTD were resolved on a 5% SDS-PAGE gel. The gel images were developed by autoradiography and scanned by Molecular Dynamics Image Scanner Storm 860 in the phosphor screen mode. Data were quantitatively analyzed by ImageQuant software.

Tissue Culture and Transfections: Human 293, COS7 and CV1 cells were grown at 37° C. in DMEM supplemented with 10% normal calf serum (BRL). Sub-confluent cells were transfected in 6 well tissue culture dishes using Effectene (Qiagen) according to the manufacturer's instructions. Reporter and activator plasmids (100 ng each) and Flag SCP1 (80 ng) or its mutant were used per well. For T3 and PPARγ transtections, 20 ng RXR plasmid was also added. The amount of ligands used are as follows: 100 nM T3, 1 μM PPARK609843 (Ligand Pharmaceuticals), 100 nM dexamethasone. LMX1 and E47 expression plasmids (100 ng) were cotransfected with 100 ng of the rat insulin promoter-luciferase reporter construct with the indicated concentrations of SCP1 and phosphatase-minus SCP1 expression plasmids. The total amounts of transfected DNA was kept constant by the addition of empty vector. Cells were harvested 48 hrs after transfections and cellular extracts were assayed for luciferase activity using Luciferase Assay System (Promega) according to the manufacturer's instructions.

Immunofluorescence: Cells grown on coverslip were fixed in 2% paraformaldehyde, neutralized and blocked using 2.5% FCS/PBS. Rabbit polyclonal IgG 6703 was used at 1:100 dilution, followed by goat anti-rabbit IgG H+L chains conjugated to Alexa Fluor 488 (1:250). Mouse anti EEA1 was. used at 1:1000 followed by goat anti-mouse IgG conjugated to Alexa Fluor 594 (1:2500). Omission of primary antibodies was used as negative control. The coverslips were viewed using the Zeiss Axiophot which is equipped with a Hamamatsu Orca ER firewire camera that runs on Improvision Openlab 3.0.9 software.

Immunoprecipitations: For immunoprecipitation experiments, 75% confluent COS7 cells from a 10 cm dish were harvested in lysis buffer (PBS containing 1% NP40, 1 mM DTT and protease inhibitors). Lysates were incubated with 20 μl sepharose-conjugated anti-SCP1 (6703) IgG at 4° C. for 6 hr. Beads were washed with PBS and the complexes were evaluated by western blotting using specific anti-RNAP II antibodies. Rabbit anti-SNX1 antibody was used as control IgG.

Northerns and in-situs: Multiple tissue blot (Clontech no. 636818) was used to determine the distribution of human SCP isoforms in adult tissues. Specific SCP1 cDNA was prepared by PCR, labeled using AlkPhos Labeling kit (Amersham) and used as probe. The same blot was stripped and rehybridized with specific SCP2 and SCP3 cDNA probes. The prehybridization, hybridization, washing and deprobing were done according to manufacturer's instructions (Clontech no. 636831). β-actin was used as internal control

In-situ Co-inmiunoprecipitation and ChIP: For Co-IP experiments, 293 cells were harvested in lysis buffer (PBS containing 1% NP40, 1 mM DTT and protease inhibitors) and incubated with either anti-SCP or anti-REST/NRSF antibodies overnight at 4° C. 15 μl of Protein A/G-Sepharose was then added to the lysates and incubated at 4° C. for 3 h. Beads were washed with PBS and the complexes were analyzed by Western blotting using either anti-REST/NRSF or anti-SCP antibodies.

ChIP was performed according to a modification of the method of Spencer et. al. (Methods 31:67-75, 2003). About 0.7×10⁶ cells were used for each ChIP experiment. Cells were crossed-linked with 1% formaldehyde for 30 min, washed twice with cold PBS, resuspended in lysis buffer (1%SDS, 10 mM EDTA, 50 mM Tris-HCl pH 8.0, 1× protease inhibitor cocktail (Roche)) and sonicated for 15s pulses at 40% with a Braun-Sonic sonicator. The lysates are clarified by centrifugation at 10000 rpm for 10 min at 40° C. in a microcentrifuge. One-tenth of the total lysate was used as input control of genomic DNA. Supernatants were collected and diluted in buffer (1% TritonX-100, 2 mM EDTA, 150 mM NaCl, 20 mM Tris-HCl pH 8.0, protease inhibitor cocktail) followed by immunoclearing with 1 μg salmon sperm. DNA, 10 μl rabbit IgG and 20 μl protein A/G-sepharose (Santa Cruz Biotechnology) for 1 h at 4° C. Immunoprecipitation was performed overnight at 4° C. with 2 μg of each specific antibody. Precipitates were washed sequentially for 10 min each in TSE1 buffer (0.1% SDS, 1% Triton-X100, 2 m M EDTA, 150 mM NaCl, 20 mM Tris-HCl pH 8.0), TSE2 (TSE 1 with 500 mM NaCl) and TSE3 (0.25M LiCl, 1% NP40, 1% deoxycholate, 1 mM EDTA, 10 mM Tris-HCl pH 8.0). Precipitates were then washed twice with TE buffer and extracted with 1% SDS containing 0.1M NaHCO3. Eluates were pooled and heated at 65° C. for 6 h to reverse the formaldehyde cross-linking. DNA fragments were purified with Qiagen Qiaquick spin kit. For PCR, 1 μl of a 25 μl DNA extraction was used.

Anti-Rest Ab (P18 and C15), anti-HP1 (D15), anti-HDAC1(H11) anti-MeCP2(H300) are from Santa Cruz Biotechnology Inc. Anti-SCP1 was prepared by immunizing rabbits using GST-SCP1 as antigen.

Inhibition of SCP expression by siRNA: S2 cells were propagated in 1× Schneider's Drosophila media/10% FBS, at room temperature. For dsRNA production, individual DNA fragments approximately 700 bp in length, spanning nt 121-853 of dSCP1 to be “knocked out” were amplified by using PCR. Each primer used in the PCR contained a 5′ T7 RNA polymerase binding site (GAATTAATACGACTCACTATAGGGAGA). The PCR products were purified by using MicroSpin S-400 columns (Pharmacia). The purified PCR products were used as templates for transcription using a MEGASCRIPT kit (Ambion). The dsRNAs were annealed by incubation at 65° C. for 30 min followed by slow cooling to room temperature The dsRNA products were ethanol-precipitated and resuspended in water. Concentration of dsRNA was determined using a spectrophotometer at OD₂₆₀. To induce RNA interference, S2 cells were diluted to a final concentration of 1×10⁶ cells/ml in Drosophila expression system (DES) serum-free medium (Invitrogen). One milliliter of cells was plated per well of a six-well cell culture dish (Corning). 12 μg dsRNA was added directly to the cells. This was followed immediately by vigorous agitation. The cells were incubated for 30 min at room temperature followed by addition of 2 ml of 1× Schneider's media containing FBS. The cells were incubated for an additional 3 days to allow for turnover of the target protein.

Quantitative RT-PCR: Total RNA from S2 cells was. prepared using RNeasy kit (Qiagen). RNA was DNase 1 treated after which the RNA concentration was determined at OD₂₆₀ mm with a spectrophotometer. Primer concentrations for the RT-PCR were optimized to yield the lowest threshold cycle (Ct) and maximum Rn while minimizing non-specific amplification. One μg of total RNA was used for each RT-PCR using the ABI Prism 7700 Sequence Detection System and SYBr Green chemistry according to manufacturer's instructions (number 4310179). To quantitate the target sequence amounts, serial dilutions (0-10 ng) of pcDNA3-SCP1 was used as template to generate a standard curve. The Ct values of standard template was plotted against the log of the corresponding copy number. The standard curve was then used to determine the amounts of the target sequences in the unknown samples, achieved by determining the corresponding Ct values. The entire process of determining the Ct values and constructing the standard curve(s) was performed as part of the data analysis using the SDS v.1.7 software. For each sample, the amplification plot and the corresponding dissociation curves were examined.

P19 Differentiation: The P19 cultured cell-lines used include a) P19 stably expressing flag-hSCP1; b) P19 stably expressing flag-hSCP1(D96E, D98N) phosphatase-inactive mutant; P19 stably expressing .EGFP; and d). P19 stably expressing REST/NRSF.

These cell lines were maintained in α-MEM/10%FBS with 400 mg/ml G418. To induce neuronal differentiation, P19 cells were allowed to aggregate in bacterial grade petri-dishes (Fisher) at a seeding density of 1×10⁵cells/ml in the presence of 1×10⁻⁶M all-trans retinoic acid. After 3 days of aggregation, cells were dissociated into single cells by 0.05% trypsin-0.53 mM EDTA (Gibco). Trypsin was removed by centrifugation and the cells were plated in 6 cm tissue culture dishes (Nunc). at a density of 1×105 cells/cm² in Neurobasal medium/N2 supplement (Invitrogen) in the presence or absence of 1 μg/ml fibronectin (Invitrogen). The cells were cultured for 5 days after which average cell numbers/field were determined. Cells were either lysed to prepare total RNA (Rneasy kit, Qiagen) or stained with TuJ1 antibody (Babco).

For immunostaining, cells were fixed for 10 mins with 4% paraformaldehyde. TuJ1 antibody was used at 1:2000 and secondary antibody used was FITC-conjuagated anti-mouse (1:3000).

RT-PCR was done using heat-stable rTth DNA Polymerase according to manufacturer's instructions (Novagen).

Embryonic stem cells, as their name suggests, are derived from embryos. Specifically, embryonic stem cells are derived from embryos that develop from eggs-that have been fertilized in vitro—in an in vitro fertilization clinic—and then donated for research purposes with informed consent of the donors. They are not derived from eggs fertilized in a woman's body. The embryos from which human embryonic stem cells are derived are typically four or five days old and are a hollow microscopic ball of cells called the blastocyst. The blastocyst includes three structures: the trophoblast, which is the layer of cells that surrounds the blastocyst; the blastocoel, which is the hollow cavity inside the blastocyst; and the inner cell mass, which is a group of approximately 30 cells at one end of the blastocoel.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

Sequences

SCP1 nucleotide sequence (SEQ ID NO:1)

| | |
|---|---|
| 1 | atggacagct cggccgtcat tactcagatc agcaaggagg aggctcgggg cccgctgcgg |
| 61 | ggcaaaggtg accagaagtc agcagcttcc cagaagcccc gaagccgggg catcctccac |
| 121 | tcactcttct gctgtgtctg ccgggatgat ggggaggccc tgcctgctca cagcggggcg |
| 181 | cccctgcttg tggaggagaa tggcgccatc cctaagaccc cagtccaata cctgctccct |
| 241 | gaggccaagg cccaggactc agacaagatc tgcgtggtca tcgacctgga cgagaccctg |
| 301 | gtgcacagct ccttcaagcc agtgaacaac gcggacttca tcatccctgt ggagattgat |
| 361 | ggggtggtcc accaggtcta cgtgttgaag cgtcctcatg tggatgagtt cctgcagcga |
| 421 | atgggcgagc tctttgaatg tgtgctgttc actgctagcc tcgccaagta cgcagaccca |
| 481 | gtagctgacc tgctggacaa atggggggcc ttccgggccc ggctgtttcg agagtcctgc |
| 541 | gtcttccacc gggggaacta cgtgaaggac ctgagccggt tgggtcgaga cctgcggcgg |
| 601 | gtgctcatcc tggacaattc acctgcctcc tatgtcttcc atccagacaa tgctgtaccg |
| 661 | gtggcctcgt ggtttgacaa catgagtgac acagagctcc acgacctcct ccccttcttc |
| 721 | gagcaactca gccgtgtgga cgacgtgtac tcagtgctca ggcagccacg gccagggagc |
| 781 | tag |

SCP1 amino acid sequence (SEQ ID NO:2)
MDSSAVITQI SKEEARGPLR GKGDQKSAAS QKPRSRGILH SLFCCVCRDD GEALPAHSGA
PLLVEENGAI PKTPVQYLLP EAKAQDSDKI CVVIDLDETL VHSSFKPVNN ADFIIPVEID
GVVHQVYVLK RPHVDEFLQR MGELFECVLF TASLAKYADP VADLLDKWGA FRARLFRESC
VFHRGNYVKD LSRLGRDLRR VLILDNSPAS YVFHPDNAVP VASWFDNNSD TELHDLLPFF
EQLSRVDDVY SVLRQPRPGS
SCP2 nucleotide sequence (nucleotides 306-1157 = SEQ ID NO:3)

| | |
|---|---|
| 1 | gccatttcct cctcttgttt tcactccgga ttctccatgt tggacccaaa ctgaggagcc |
| 61 | cggagctgcc gctgggggat cggggccggg ggcacccggg ggagccgctg cccgggccgc |
| 121 | ccgccctttg tacaggccgc ctcccttccc ggtccgggga ggaaacgaga ggggggatgt |
| 181 | gaacagctgt ggaagtcgga gtctcgggag ccggagcggg cccccgccca ggccccccag |
| 241 | cccagcccag cccgcgcgcc cgcccgtcct cccgtccagc cagcccgggc ccgcgggatt |
| 301 | gttagatgga acacggctcc atcatcaccc aggcgcggag ggaagacgcc ctggtgctca |
| 361 | ccaagcaagg cctggtctcc aagtcctctc ctaagaagcc tcgtggacgt aacatcttca |
| 421 | aggccctttt ctgctgtttt cgcgcccagc atgttggcca gtcaagttcc tccactgagc |
| 481 | tcgctgcgta taaggaggaa gcaaacacca ttgctaagtc ggatctgctc cagtgtctcc |
| 541 | agtaccagtt ctaccagatc ccagggacct gcctgctccc agaggtgaca gaggaagatc |
| 601 | aaggaaggat ctgtgtggtc attgacctcg atgaaaccct tgtgcatagc tcctttaagc |
| 661 | caatcaacaa tgctgacttc atagtgccta tagagattga ggggaccact caccaggtgt |
| 721 | atgtgctcaa gaggccttat gtggatgagt tcctgagacg catgggggaa ctctttgaat |
| 781 | gtgttctctt cactgccagc ctggccaagt atgccgaccc tgtgacagac ctgctggacc |
| 841 | ggtgtggggt gttccgggcc cgcctattcc gtgagtcttg cgtgttccac cagggctgct |
| 901 | acgtcaagga cctcagccgc ctggggaggg acctgagaaa gaccctcatc ctggacaact |
| 961 | cgcctgcttc ttacatattc caccccgaga atgcagtgcc tgtgcagtcc tggtttgatg |
| 1021 | acatggcaga cactgagttg ctgaacctga tcccaatctt tgaggagctg agcggagcag |
| 1081 | aggacgtcta caccagcctt ggggcagctg cgggcccctt agcctgccct gcttccaagc |
| 1141 | gacggccatc ccagtagggg actttcccac actgtgcctt tacgatcagc gtgacagagt |
| 1201 | agaagctgga gtgcctcacc acacggcccg gaaacagcgg gaagtaactg gaaagagctt |
| 1261 | taggacagct tagatgccga gtgggcgaat gccagaccaa tgatacccag agctacctgc |
| 1321 | cgccaacttg ttgagatgtg tgtttgactg tgagagagtg tgtgtttgtg tgtgtgtttt |
| 1381 | gccatgaact gtggccccag tgtatagtgt ttcagtgggg gagaagctga aagaccaaga |
| 1441 | ctcttcccaa gttagcttgt ctcctctcct gtcaccctaa gagccactga gttgtgtagg |
| 1501 | gatgaaract attgaagact ccattgccaa accatggcct ttcctcagtg ttgtaaggcc |
| 1561 | tatgccaagg ataaaggaag ggtatgcctt tgggtactcc aggcatacac ctttctgaaa |
| 1621 | tccttctcca gccagctgct gcagacaaaa gatcacattt ctgggaagat gagaacttgt |
| 1681 | ttccagacca gcatccagtg gccatcaggt cttgtggccc aaaggctatg cttgcctccg |
| 1741 | gctgagtgcc tgggataggc cttttctatg tctccccaag gctggggtgc tgagcctgcc |
| 1801 | ttcctcacca cctagccata gtctcaaacc tgtggggaag gaggttttct ccctgcccgg |
| 1861 | gaagaggaca gataactgat ttccgttctt ttgactgtgt tttaaaattc tctttctaaa |
| 1921 | cacagagtgt tgggcctggt ttgtttctga caaagttaca gtcctgggcc tgtaatgaat |
| 1981 | gtcggcggcg ctggggttgc agggaaaaga caaatcctca aagcgtggac gtgtgtcccc |
| 2041 | atggcttgtg gatcagctaa gctcgggatc atttccataa gtctgctttt cagggattct |
| 2101 | ctgctggtgc tggtgcaagg acttctgttc caaaggctgg gaaaaactaa gctgtcccag |
| 2161 | cccctcccat ttcttgggca gggctctttt cctgttgtgt cttcccccag ggcctgtcct |
| 2221 | gtaccgagct ctgtctgttc cagcctacat ccttcctggg tgttgctttt cctcttaagg |
| 2281 | gcctcagaac tcttgctctt cctggggtga gggggaatga gtgttcttga catgtgacag |
| 2341 | cctaatgcgc atgctttctg cctctggtaa caggagtgag tgagcccctc agacctgcac |
| 2401 | tctgggtgtc tcctgcttac aaaggttctt aatagtgaat gctttaaaat taaagtcatc |
| 2461 | acgaaatgga agttttccca gggtggaaaa taagaggaag tgctgctgta attgggagca |
| 2521 | caaggggcct cccaaaaagg agccccacct cagcatcact gccttaatcg tggcctccct |
| 2581 | ggggtgggtg gggttctctc ctccctccct ccctcctcct ggggtgggag ggcgctcctg |
| 2641 | ttcccatctc tgtgttccct ggaggcaggt atcacaaagc atttgtgaat tgctttaggt |
| 2701 | gcagggacac cacccactca ggactcttcc ccatcatccc ttccattgcc acaccctaga |
| 2761 | tccagcctca ggaactaaca agttktgaga aaagcaggtg gtagagcagc agcttcgtgc |
| 2821 | tctcagcggt ggctggctgg catttttctc tagcgttgtg gtgccacctt cccttcttgt |
| 2881 | cccaaggtta taaggccttg tctttctctt tggaatcata aagtggaaca gagtccccag |
| 2941 | aactcatgtg ghcatttccg acagcatcac tccccggtgc ctatggggtc ccggtgtacc |
| 3001 | taaagggaga aggaccccat gtgctagcca gaaatatact gtctcttgaa ggaaagcagg |
| 3061 | agctcagact cttagagcca gctgtggctt cggacccaag gcctgaccta ggctgctatc |
| 3121 | ctaatattgg aggaggggcc tctcttccaa gccccaccct aagggttagc ccttggacaa |
| 3181 | atcttgtgcc gtctaggccc agccaggctt ttctgactaa ataagcaata agaggctcta |
| 3241 | agctgactga gttgcaagga ccctttccgc cctcccttgg atctccatgt ttctccagat |
| 3301 | ggcggaagag catgtgccac cccctttcct aacagacttg tccaagtgct tggcgtggga |
| 3361 | cccatgacca aagcccagga tggcttggtg ggagtgtccc tgctgcatct gcatgaagcc |
| 3421 | cctgcttttt aggcctcact cccatcagaa ccctgcctgc ccacctgcaa ctccccccca |
| 3481 | acaatgccat tcccacttgc cccagagaag ctactcggcc aaacctagcc agggtctgtt |
| 3541 | cttgtggacc agagccagcc tagtcattat ttgctgtcgg gtttccagtt tcaccgtgtg |
| 3601 | ttagggtgag ggatgattgt aaaatttgct cctcaaagga atcaggccag actcaatttt |
| 3661 | gggagggcaa gacagggagg aggccgcttc atcccagact ctcttctagg gcttcccacc |
| 3721 | atcagcccct cccacttgag actggtcttt gggaggcaat aggccaccat gcctggtcag |
| 3781 | caccaattca agccatgcca ggaatctgcc tacctgccag gttcagttct tttaaggtgc |
| 3841 | ctcttcaggg acacagtgtg tctctctgat tgggcttcta aatcaaaagc ctgatgttcg |
| 3901 | tgtccctctc atagggggag ctttggacac aggaccagtt tggaaaaggg tcaggtaagg |
| 3961 | gtttccactc tgcacattgt agagggaaca ctctgtaggc ccatgggtcc cttactagag |
| 4021 | aggttgagtg aatttgcctt cagttaacat gggaccttct gtttagcttc ctcttgcttc |
| 4081 | ccaaagattt taagcatttt gtaaatgtat aaactcacct ctggtaacag tggcccagac |
| 4141 | gctgctttgt gctaaaagca tgggaaatgt aaaggcagtc tttctctggg aaatggatgc |
| 4201 | tattctattc tgctgcccct acctgttcct gaggcctcat ttagaaagaa aatcccctca |
| 4261 | gaaggctgtc tggcacccag tgtcctagcc aggccaagta tatgagaaag gtaagtccat |
| 4321 | tttccccttc aggtcctcag tggattactt aaccactgct gtccctcggt ccctttttcc |
| 4381 | taaacgggtt tagttctgtc ttttttctcc ttttttctaa atgctggtaa atatttacat |
| 4441 | tcagccaggg aagaggaggc cagaggtcgg gccagctgcc ccattctttt aacgttgtag |
| 4501 | ggcctgccca tggagcggac cctcctcttt gggcctcgtg agcttttttg cttatcatgt |
| 4561 | tccatttcgt gccgctttcc cccttcaaga tgccatttgg agggtagggg atctgcttcc |
| 4621 | cactgtgact gggctatggg attctgacta ccttgcttac agattcatgg tttgataaat |
| 4681 | ttgttgtatt ccaaaacttg aaatgcagga cgccattaag tgtctgttta tatttttgga |
| 4741 | atatttgtat tacttacaat taattaataa aagtgggttt aaaaaacctt tccaggaaaa |
| 4801 | aaaaaaaaaa aaaaaaaaaa aaaaaaaaaa aaa |

SCP2 amino acid sequence (SEQ ID NO:4)
MEHGSIITQARREDALVLTKQGLVSKSSPKKPRGRNIFKALFCC
FRAQHVGQSSSSTELAAYKEEANTIAKSDLLQCLQYQFYQIPGT
CLLPEVTEEDQGRICVVIDLDETLVHSSFKPINNADFIVPIEIE
GTTHQVYVLKRPYVDEFLRRMGELFECVLFTASLAKYADPVTDL
LDRCGVFRARLFRESCVFHQGCYVKDLSRLGRDLRKTLILDNSP
ASYIFHPENAVPVQSWFDDMADTELLNLIPIFEELSGAEDVYTS
LGAAAGPLACPASKRRPSQ
SCP3 nucleotide sequence (nucleotides 1-798 = SEQ ID NO:5)

| | |
|---|---|
| 1 | atggacggcc cggccatcat cacccaggtg accaacccca aggaggacga gggccggttg |
| 61 | ccgggcgcgg gcgagaaagc ctcccagtgc aacgtcagct taaagaagca gaggagccgc |
| 121 | agcatcctta gctccttctt ctgctgcttc cgtgattaca atgtggaggc ccctccaccc |
| 181 | agcagcccca gtgtgcttcc gccactggtg gaggagaatg gtgggcttca gaagccacca |
| 241 | gctaagtacc ttcttccaga ggtgacggtg cttgactatg gaaagaaatg tgtggtcatt |
| 301 | gatttagatg aaacattggt gcacagttcg tttaagccta ttagtaatgc tgattttatt |
| 361 | gttccggttg aaatcgatgg aactatacat caggtgtatg tgctgaagcg gccacatgtg |
| 421 | gacgagttcc tccagaggat ggggcagctt tttgaatgtg tgctctttac tgccagcttg |
| 481 | gccaagtatg cagaccctgt ggctgacctc ctagaccgct ggggtgtgtt ccgggcccgg |
| 541 | ctcttcagag aatcatgtgt ttttcatcgt gggaactacg tgaaggacct gagtcgcctt |
| 601 | gggcgggagc tgagcaaagt gatcattgtt gacaattccc ctgcctcata catcttccat |
| 661 | cctgagaatg cagtgcctgt gcagtcctgg ttcgatgaca tgacggacac ggagctgctg |
| 721 | gacctcatcc ccttctttga gggcctgagc cgggaggacg acgtgtacag catgctgcac |
| 781 | agactctgca ataggtagcc ctggcctctg cctgcctccc gcctgtgcac tctggaacct |
| 841 | ctggcctcag gggacctgc |

SCP3 amino acid sequence (SEQ ID NO:6)
MDGPAIITQVTNPKEDEGRLPGAGEKASQCNVSLKKQRSRSILS
SFFCCFRDYNVEAPPPSSPSVLPPLVEENGGLQKPPAKYLLPEV
TVLDYGKKCVVIDLDETLVHSSFKPISNADFIVPVEIDGTIHQV
YVLKRPHVDEFLQRMGQLFECVLFTASLAKYADPVADLLDRWGV
FRARLFRESCVFHRGNYVKDLSRLGRELSKVIIVDNSPASYIFH
PENAVPVQSWFDDMTDTELLDLIPFFEGLSREDDVYSMLHRLCNR
SCP1 214 nucleotide sequence (nucleotides 1-642 = SEQ ID NO:7)

| | |
|---|---|
| 1 | atgatgggga ggccctgcct gCtcacagcg gggcgcccct gcttgtggag gagaatggcg |
| 61 | ccatccctaa ggcagacccc agtccaatac ctgctccctg aggccaaggc ccaggactca |
| 121 | gacaagatct gcgtggtcat cgacctggac gagaccctgg tgcacagctc cttcaagcca |
| 181 | gtgaacaacg cggacttcat catccctgtg gagattgatg gggtggtcca ccaggtctac |
| 241 | gtgttgaagc gtcctcacgt ggatgagttc ctgcagcgaa tgggcgagct ctttgaatgt |
| 301 | gtgctgttca ctgctagcct cgccaagtac gcagacccag tagctgacct gctggacaaa |
| 361 | tggggggcct tccgggcccg gctgtttcga gagtcctgcg tcttccaccg ggggaactac |
| 421 | gtgaaggacc tgagccggtt gggtcgagac ctgcggcggg tgctcatcct ggacaattca |
| 481 | cctgcctcct atgtcttcca tccagacaat gctgtaccgg tggcctcgtg gtttgacaac |
| 541 | atgagtgaca cagagctcca cgacctcctc cccttcttcg agcaactcag ccgtgtggac |
| 601 | gacgtgtact cagtgctcag gcagccacgg ccagggagct agtgagggtg atggggccag |
| 661 | gacctgcccc tgaccaatga tacccacacc tcctcccagg aagactgccc aggcctttgt |
| 721 | taggaaaacc catgggccgc cgccacactc agtg |

SCP1 214 amino acid sequence (SEQ ID NO:8)
mmgrpcllta grpclwrrma pslrqtpvqy llpeakaqds dkicvvidld etlvhssfkp
vnnadfiipv eidgvvhqvy vlkrphvdef lqrmgelfec vlftaslaky adpvadlldk
wgafrarlfr escvfhrgny vkdlsrlgrd lrrvlildns pasyvfhpdn avpvaswfdn
msdtelhdll pffeqlsrvd dvysvlrqpr pgs
SCP1 D96E, D98N mutant nucleotide acid sequence (SEQ ID NO:9)

| | |
|---|---|
| 1 | atggacagct cggccgtcat tactcagatc agcaaggagg aggctcgggg cccgctgcgg |
| 61 | ggcaaaggtg accagaagtc agcagcttcc cagaagcccc gaagccgggg catcctccac |
| 121 | tcactcttct gctgtgtctg ccgggatgat ggggaggccc tgcctgctca cagcggggcg |
| 181 | cccctgcttg tggaggagaa tggcgccatc cctaagaccc cagtccaata cctgctccct |
| | g |
| 241 | gaggccaagg cccaggactc agacaagatc tgcgtggtca tcgaactgaa cgagaccctg |
| 301 | gtgcacagct ccttcaagcc agtgaacaac gcggacttca tcatccctgt ggagattgat |
| 361 | ggggtggtcc accaggtcta cgtgttgaag cgtcctcatg tggatgagtt cctgcagcga |
| 421 | atgggcgagc tctttgaatg tgtgctgttc actgctagcc tcgccaagta cgcagaccca |
| 481 | gtagctgacc tgctggacaa atggggggcc ttccgggccc ggctgtttcg agagtcctgc |
| 541 | gtcttccacc gggggaacta cgtgaaggac ctgagccggt tgggtcgaga cctgcggcgg |
| 601 | gtgctcatcc tggacaattc acctgcctcc tatgtcttcc atccagacaa tgctgtaccg |
| 661 | gtggcctcgt ggtttgacaa catgagtgac acagagctcc acgacctcct ccccttcttc |
| 721 | gagcaactca gccgtgtgga cgacgtgtac tcagtgctca ggcagccacg gccagggagc |
| 781 | tag |

SCP1 D95E, D9N mutant amino acid sequence (SEQ ID NO:10)
MDSSAVITQI SKEEARGPLR GKGDQKSAAS QKPRSRGILH SLFCCVCRDD GEALPAHSGA
PLLVEENGAI PKTPVQYLLP EAKAQDSDKI CVVIELNETL VHSSFKPVNN ADFIIPVEID
GVVHQVYVLK RPHVDEFLQR MGELFECVLF TASLAKYADP VADLLDKWGA FRARLFRESC
VFHRGNYVKD LSRLGRDLRR VLILDNSPAS YVFHPDNAVP VASWFDNMSD TELHDLLPFF
EQLSRVDDVY SVLRQPRPGS
SCP1 214 D48E, D50N mutant nucleotide acid sequence (SEQ ID NO:11)

| | |
|---|---|
| 1 | atgatgggga ggccctgcct gctcacagcg gggcgcccct gcttgtggag gagaatggcg |
| 61 | ccatccctaa ggcagacccc agtccaatac ctgctccctg aggccaaggc ccaggactca |
| | g |
| 121 | gacaagatct gcgtggtcat cgaactgaac gagaccctgg tgcacagctc cttcaagcca |
| 181 | gtgaacaacg cggacttcat catccctgtg gagattgatg gggtggtcca ccaggtctac |
| 241 | gtgttgaagc gtcctcacgt ggatgagttc ctgcagcgaa tgggcgagct ctttgaatgt |
| 301 | gtgctgttca ctgctagcct cgccaagtac gcagacccag tagctgacct gctggacaaa |
| 361 | tggggggcct tccgggcccg gctgtttcga gagtcctgcg tcttccaccg ggggaactac |
| 421 | gtgaaggacc tgagccggtt gggtcgagac ctgcggcggg tgctcatcct ggacaattca |
| 481 | cctgcctcct atgtcttcca tccagacaat gctgtaccgg tggcctcgtg gtttgacaac |
| 541 | atgagtgaca cagagctcca cgacctcctc cccttcttcg agcaactcag ccgtgtggac |
| 601 | gacgtgtact cagtgctcag gcagccacgg ccagggagct ag |

SCP1 214 D48E, D50N mutant amino acid sequence (SEQ ID NO:12)
MMGRPCLLTA GRPCLWRRMA PSLRQTPVQY LLPEAKAQDS DKICVVIELN ETLVHSSFKP
VNNADFIIPV EIDGVVNQVY VLKRPHVDEF LQRMGELFEC VLFTASLAKY ADPVADLLDK
WGAFRARLFR ESCVFHRGNY VKDLSRLGRD LRRVLILDNS PASYVFHPDN AVPVASWFDN
MSDTELHDLL PFFEQLSRVD DVYSVLRQPR PGS
SCP1 nucleic acid sequence on chromosome 2 (SEQ ID NO:13)
ctggagcgcg gcaggaaccc ggcccggccc gcctcccagt ccgcctagcc gcgccggtcc
cagaagtggc gaaagccgca gccgagtcca ggtcacgccg aagccgttgc ccttttaagg
gggagccttg aaacggcgcc tgggttccat gtttgcatcc gcctcgcggg aaggaaactc
catgttgtaa caaagtttcc tccgcgcccc ctccctcccc ctccccccta gaacctggct
cccctcccct ccggagctcg cggggatccc tccctcccac ccctcccctc ccccccgcgc
cccgattccg gccccagccg ggggggaggc cgggcgcccg ggccagagtc cggccggagc
ggagcgcgcc cggccccatg gacagctcgg ccgtcattac tcagatcagc aaggaggagg
ctcggggccc gctgcggggc aaaggtaccg gggctgcggg gagggggccg aagccggggc
gccgtgggag gagagaaggg gccgggatct tccccagggg agccgccgcc gccgccccgg
gcggccgcct tagctgtgcc cgaagctccc agcccgagag ggagcaggga gagagtttga
actcagagga ggctcagaga cgcggggcgg ggcctggcgc ctttggggcg ctcctgtccg
ctcgaggtga ggaaactgag gcaggaatag agagggaact ccttcggggg tttcctggca
ggcattgcgt ggtgcatggg cgccccccca ccattggcgc caatggggct gtgagatggg
ggagctgagg agggcgccta tgggccaccc gctgagactc cgccccaccc cccaccccca
cccccccggg ctgcggtccg gtagggtctt gggagggggc gccgaggtga cagcaggctg
gggaggcttg gagggatctc ccgccaacac acagctacgt tccccacaaa cttcgcgtca
cgcgtggagg cgccgacccc ctcggaggca cagagaggac ggccggcact tccaagagtc
gcttggcgcc cgcggggaga gtcgtgcgcc tagtgggcac gcaccacccc gcaaagcctc
gccgccccga cgaggctgcg tcccccagcg tggctgggcc ggggtggggg ggtctgtctt
ctccttttcc ccgtgtggac ctcaggatct ggacgctgcc cccaggtctg cccaccctcg
cctgggtctg gctgccccgg aactgagggc aaggtggaaa ggctagttgc agggggccgg
aggggggtgg ggtgggaggg gtatctgtca atcaggctgc tgggctccag gtcggaggtc
tgggcggggc agggcaaaca gatggccact ggacactggc cccaggccgc gggactgcac
ccctgcctct gggcccagcc gcagtgagga cttcgtaccc acgggggtgg agaggatgga
gggagggcag gggtggactg ccctgggtcc caggccctgg ctgtcctgag caggggtgct
caggtaaggt ggggtcagga ggcaccgcaa tggggctgat cagcagcagt catggaggct
gtgagaggca gggagagagc accccaggac ctccttctcc aggccacgca ctccctatgt
gggcgcctta atacctgcta gacctatttg tctgggagct gcaggagcct tggagttgat
tgtggagccc tgacaggggc gtttcagaga aagtcaggag ctgccttcgt gtgtctggat
gaaggggcca cggcaagatc ctcctggccc aggggttcac acctgggcac acatgcagga
ttctgcaggc cagtgtgcac cgagcctcca acttgtgcct ccctacttca ggtgaccaga
agtcagcagc ttcccagaag ccccgaagcc ggggcatcct ccactcactc ttctgctgtg
tctgccggga tgatggggag gccctgcctg ctcacagcgg ggcgcccctg cttgtggagg
agaatggcgc catccctaag gtgcgtgggg gccaggtggg gccacggggg cacctggact
cagtcttcag ggctttaggg gaaggggctc ctgactgagc ttttcaggat ggacttgcag
acctgaaagt gcagagtagg agggtggcag cctcccctgc caggccctgc ccactgtggg
gaaactgaat tctccctcat aagtggaagc ttttttctac cttggttttt agagaggtct
caaagagcca agaggcctac ccaagcccta gagctggcag gggcaaagct gggaaggggg
aagtatctgt tcctggggcc tggggttcct ctggagacgg ctagggggag aagcctgcgt
gggaggaagg accaggcccg gagagaggca ccccagccag ccccgccctc cctacagcag
accccagtcc aatacctgct ccctgaggcc aaggcccagg actcagacaa gatctgcgtg
gtcatcgacc tggacgagac cctggtgcac agctccttca aggtgggccc tgctcaacag
ccctcagccc gggtctcggg gggcatcccc caccctggcc tgggagggag gtgtgtgctg
gaccccatgc cctggggctc ctcctccaac tccagcagct cttttccccc cacagccagt
gaacaacgcg gacttcatca tccctgtgga gattgatggg gtggtccacc aggtgagggc
caggaagagg cagtggtggg cttggcatct gcctccagac cctaggctct tcccaccaat
ccggagcgcc tcggatggga attggataca tgtggaatgt cagaggccca gagagggtgt
gagacttgtc ccaaagtcac acagaacctc aagggcttgt gctgactcca agcctgcaga
gtgggctcct cctctaggct cccccgtgct gtgctccctc gccccaccct gcccgggacc
cagttcaagt aattcaggat aggttgtgtg ctgtccagcc tgttctccat tacttggctc
ggggaccggt gccctgcagc cttggggtga gggggctgcc cctggattcc tgcactaggc
tgaggttgag gcaggggaag ggattgggaa ttagggacct cgtgaggtag gactggccag
tggagtggaa gttttgatcg ttttctggcg gggggtgggt acagtttccc cagcagtggt
cagggtagct ggccaagcgg agcctgcggg cccagtctcc ttcctgtgcg cctctgcctc
cctggcccat gccctgccag ccctcggcca cccccacact gccccactgg cccgcagccc
cctcactggc ccgcccccca ggtctacgtg ttgaagcgtc ctcatgtgga tgagttcctg
cagcgaatgg gcgagctctt tgaatgtgtg ctgttcactg ctagcctcgc caaggtgagc
cccacagggg tcccggggca accctgccct cctacctacc tcccgcatgc agcccagtga
acctgcgggc cccaggatga cccacctcct gctcccagta cgcagaccca gtagctgacc
tgctggacaa atggggggcc ttccgggccc ggctgtttcg agagtcctgc gtcttccacc
gggggaacta cgtgaaggac ctgagccggt tgggtcgaga cctgcggcgg gtgctcatcc
tggacaattc acctgcctcc tatgtcttcc atccagacaa tgctgtgagt gcgggctgga
ctgggactgg gacaggagct gagacccagg aaggggtcag tccattcagg ccaccttggc
ctcttggatc cccagttggg gggtgggtgc cctcccagtc cttcctgcat tcattgcctg
tgcctgccgc ccactcccct catccacctg ccctgtagcc atatggtctt ttcccctcgc
acaaagcaga gcatctgcca tgcacagggg cccccacagg gcaacggagt ttggaaagtt
tcaatttttc gaattgccag ttgtgaccta ctgatggccc acagaattaa tttagtgggt
tctgattggg aattttaaca aaatgaaata gaatagaaaa tatccggtcg ggtgcagtgg
ctcatgcctg taatcccagc actttgggaa gctgaggtgg gcaggtagct gagcccagta
gttcaagacc agcctcggca acatagtgaa accttatgtc tacaaaaaat acaaaaacta
gccaggcgtg gtggcgcatg cctggagtcc cggctatgca gaaggctgag gtaggagtat
cgcttgagcc ctggaggcag aggctgtggt gagccaagat tgtgccactg cactctagcc
tgggcaacag agcaagaccc tgcctcaaaa aaaaaaaaaa gtatccaagt gcttcgcaca
gataaggtta ggaattgtga agcttttgca ttgttacgtt ataaatgtgt tttcctgggg
attgctgtca aaaaagtttg aacactgtgg gtgaggggtt ttcagaaact gcatgatctg
agtagtggct acatagggct ggcctggaaa ttctgcaccc aggaccacct gcccccctca
tcttcctaca cccacttccc caggtaccgg tggcctcgtg gtttgacaac atgagtgaca
cagagctcca cgacctcctc cccttcttcg agcaactcag ccgtgtggac gacgtgtact
cagtgctcag gcagccacgg ccagggagct agtgagggtg atggggccag gacctgcccc
tgaccaatga tacccacacc tcctcccagg aagactgccc aggcctttgt taggaaaacc
catgggccgc cgccacactc agtgccatgg ggaagcgggc gtctccccca ccagccccac
caggcggtgt aggggcagca ggctgcactg aggaccgtga gctccaggcc ccgtgtcagt
gccttcaaac ctcctcccct attctcaggg gacctggggg gccctgcctg ctgctccctt
tttctgtctc tgtccatgct gccatgtttc tctgctgcca aattgggccc cttggcccct
tccggttctg cttcctgggg gcagggttcc tgccttggac ccccagtctg ggaacggtgg
acatcaagtg ccttgcatag agccccctct tccccgccca gctttcccag gggcacagct
ctaggctggg aggggagaac cagcccctcc ccctgcccca cctcctccct tgggactgag
agggccccta ccaacctttg cctctgcctt ggagggaggg gaggtctgtt accactgggg
aaggcagcag gagtctgtcc ttcaggcccc acagtgcagc ttctccaggg ccgacagctg
agggctgctc cctgcatcat ccaagcaatg acctcagact tctgccttaa ccagccccgg
ggcttggctc ccccagctct gagcgtgggg gcataggcag gacccccctt gtggtgccat
ataaatatgt acatgtgtat atagattttt aggggaagga gagagggaag ggtcagggta
gagacacccc tcccttgccc ctttcctggg cccagaagtt ggggggaggg agggaaagga
tttttacatt ttttaaactg ctattttctg aatggaacaa gctgggccaa ggggcccagg
ccctgtcctc tgtccctcac acccctttgc tccgttcatt cattcaaaaa aacatttctt
gagcaccttc tgtgcccagc atatgctagg cccaccagct aagtgtgtgt ggggggtctc
tacgccagct catcagtgcc tccttgccca tccttcaccg gtgcctttgg gggatctgta
ggaggtggga ccttctgtgg ggtttgggga tctccaggaa gcccgaccaa gctgtcccct
tcccctgtgc caacccatct cctacagccc cctgcctgat cccctgctgg ctgggggcag
ctcccaggat atcctgcctt ccaactgttt ctgaagcccc tcctcctaac atggcgattc
cggaggtcaa ggccttgggc tctccccagg gtctaacggt taaggggacc cacataccag
tgccaagggg gatgtcaagt ggtgatgtcg ttgtgctccc ctcccccaga gcgggtgggc
ggggggtgaa tatggttggc ctgcatcagg tggccttccc atttaagtgc cttctctgtg
actgagagcc ctagtgtgat gagaactaaa gagaaagcca gacccctatc ctgcttctgt
ggttattgcg ggggacttca gcaagtgggg tgtgtgcctt gcacctgcgg ctgccgtggg
cccccccccc gcttcagcac acctagaggg ctgttggtgg agggaggggc tgcccggccc
tcgacacttc aggtgggaag ggcagcgtca gagcacaaat ttgagcctcc aggctgtgct
cgtctacgtc ttcccgcctc gggtatgtgg tctgcaaaat ggagatgtgc cctattggca
ggactaatta agtgcctgga cacagacgac aggatactag tagctggaaa gcaaaattcg
aaggcctggg taggggcagt cctggaatgc ggcgggggag ggggcgtggc ctctgccctg
gagcagaggg gcggggcttg tgcggctccg aaggcagagg cggggagcgg ggcgaggctc
tgggtggagg ctccagcggc agaacttgtt ggcctgggtg cggcgggctc cggcgcctgg
ctctgccggg cggcctgggt ggggccggcg ccggggctcg gccccccccg cccctctgcg
gcctctgagc agccattggc cgcgcccccg ccccacttcc cgccccgccc cgcgtccggg
aggcacttcc tttgcgaaac cgcgcggccc caggcgccgg caggaaatgc cctcccgccg
tccccagcca gcctttgctt gcttcccacg ccagccgcta gaggcctccc tgtcctcgcg
gacgcaggaa ctccccgggg gctggaaaga tggggcccac ctcactcacc cctttcccgg

It will be readily apparent to one skilled in the art that varying substitutions and modifications may be made to the invention disclosed herein without departing from the scope and spirit of the invention. Thus, such additional embodiments are within the scope of the present invention and the following claims.

TABLE 1

| | | | |
|---|---|---|---|
| Name of gene | 5′ sequence | 3′ sequence | Accession no. |

Fly primers for qRT-PCR

| | | | |
|---|---|---|---|
| SCP1 | 5′ atgggcgaactatacgagtgcgttc 3′ | 5′ cttgtctgctgctggttcaacatgg 3′ | CG5830 |
| GAPDH | 5′ atcaacgacaacttcgagatcgtcg 3′ | 5′ gcggttggagtagccaaactcgttg 3′ | CG12055 |
| ribosomal | 5′ atgtcgctcttgcaaaaactaagc 3′ | 5′ ttataggatatcttcgattttcggc 3′ | CG5497 |
| protein S35 | | | |
| beta-actin | 5′ tgaagatcctcaccgagcgcggcta 3′ | 5′ gaccggactcgtcatactcctgcttg 3′ | NM_079486 |
| Na Channel II | 5′ cagctggtgcgggagtacggcttcc 3′ | 5′ tgcgcagctcgcccatgtagacctg 3′ | CG9071 |
| synapsin | 5′ gagctgtcgttgagctttggcg 3′ | 5′ cgcgtggattggggaagaaggtc 3′ | CG3985 |
| cholineAcetyl- | 5′ actgggcctattactactggctc 3′ | 5′ ccgtaaaaccgcgcgcattaaagt 3′ | CG32848 |
| Transferase | | | |
| ELAV | 5′ caacgaagccgagcgagccatccag 3′ | 5′ tggtcatggtcacgaatccgaatc 3′ | CG4396 |
| beta-tubulin | 5′ gcaacaactgggccaagggtcattac 3′ | 5′ cttggcatcgaacatctgctgggtcag 3′ | CG9277 |
| Neurofilament H | 5′ gccttccaagagcacgacgtacaaag 3′ | 5′ cgatcagaagtggatcgcggtcctta 3′ | CG7421 |
| peptidyl- | 5′ ctcgccaat caagtacctt gtgctgc 3′ | 5′ ccctggctgaagcagaacttcatg 3′ | CG3832 |
| glycine | | | |
| oxygenase | | | |
| myosin-light- | 5′ cttcgctcgcacctcagaaacgatc 3′ | 5′ tatggcataaaaggtgtggccattc 3′ | CG1915 |
| chain-kinase | | | |
| GCM | 5′ caacggaactaacggccgctccgag 3′ | 5′ gttctcgccatcgttgagatctgc 3′ | CG12245 |
| nMDAR | 5′ ctcgccattgttctcctggtgg 3′ | 5′ cgtacatgaggtagaccctgga 3′ | CG14793 |

Mouse Primers for RT-PCR

| | | | |
|---|---|---|---|
| SCP1 | 5′ cggccgtcattactcagatcagcaagg 3′ | 5′ gcagtgaacagcacacattcaaagagct 3′ | AY028804 |
| GAPDH | 5′ tccaccaccctgtgttgctgta 3′ | 5′ accacagtccatgccatcac 3′ | NM_008084 |
| ngn1 | 5′ catctctgatctcgactgctccagcag 3′ | 5′ gggtcagagagtggtgatgccacagtg 3′ | NM_010896 |
| beta-tubulin | 5′ tgccctcacccaaggtctctgacactgtgg 3′ | 5′ cttgaacagctcctggatggcagtgctg 3′ | NM_023716 |
| stra13 | 5′ ctgtggccatggagggaaacagtggcttcc 3′ | 5′ agaagtccaggagcagctgagggagcac 3′ | NM_016665 |
| GAD1 | 5′ gcaaccgcaggcacgactgtttacggag 3′ | 5′ agatgaccatccggaagaagttggccttgt 3′ | NM_008077 |
| nrsf | 5′ ccatcgcctgcgaaacctccccaggtaga 3′ | 5′ agccaactcagctggactctctccagcttc 3′ | NM_011263 |

Human Primers for ChIP assay

| | | | |
|---|---|---|---|
| GAD1 promoter | 5′ tgcggtttatattatcctgcacgccgggag 3′ | 5′ caccggttcgagtccccggagaggatatc 3′ | NT005403 |
| chr2q31 | | | |
| GAD1 3′ gene | 5′ ggagccctatgcagggtaagggaataa 3′ | 5′ gggctttgatttttggagccaccttgtg 3′ | NT005403 |
| chr2q31 | | | |
| GRIN 2A | 5′ aactatttctgggtcactccttagacac 3′ | 5′ gctgggaggaatgctttctaatgcatttg 3′ | NT010393 |
| promoter chr16 | | | |
| SCN2 promoter | 5′ ctggataagttactgaagagtgggctttgg 3′ | 5′ cagacgacaagttacatgcaacatg 3′ | NT005403 |
| chr.2q23 | | | |

What is claimed is

1. An isolated nucleic acid molecule selected from the group consisting of:
a) a nucleic acid molecule consisting of a nucleotide sequence which is at least 80% identical to the nucleotide sequence of SEQ ID NO:1, 3, 5, 7, 9 or 11;
b) a nucleic acid molecule comprising a nucleotide sequence which is at least 80% identical to the-nucleotide sequence of SEQ ID NO:1, 3, 5, 7, 9 or 11;
c) a nucleic acid molecule which encodes a polypeptide consisting of the amino acid sequence of SEQ ID NO:2, 4, 6, 8, 10 or 12;
d) a nucleic acid molecule which encodes a polypeptide comprising the amino acid sequence of SEQ ID NO:2, 4, 6, 8, 10 or 12;
e) a nucleic acid molecule which encodes a polypeptide comprising the amino acid sequence of SEQ ID NO:2, 4, 6, 8, 10 or 12 with 0 to 50 conservative amino acid substitutions; and
f) a nucleic acid molecule which encodes a naturally occurring allelic variant of a polypeptide comprising the amino acid sequence of SEQ ID NO:2, 4, 6, 8, 10 or 12 wherein the nucleic acid molecule hybridizes to a nucleic acid molecule consisting of SEQ ID NO: 1, 3, 5, 7, 9 or 11, or a complement thereof, under stringent conditions.

2. An isolated nucleic acid molecule selected from the group consisting of:
a) the cDNA deposited with ATCC as Accession Number BE300370;
b) the cDNA deposited with ATCC as Accession Number AL520011; and
c) the cDNA deposited with ATCC as Accession Number AL520463,
or a complement thereof.

3. A nucleic acid molecule comprising the nucleotide sequence of SEQ ID NO:1, 3, 5 , 7, 9 or 11.

4. A nucleic acid molecule consisting of the nucleotide sequence of SEQ ID NO:1, 3, 5, 7, 9 or 11.

5. The isolated nucleic acid molecule of claim 1, wherein the nucleotide sequence is at least 90% identical to SEQ ID NO:1, 3, 5, 7, 9 or 11.

6. The isolated nucleic acid molecule of claim 1, wherein the nucleotide sequence is at least 95% identical to SEQ ID NO:1, 3, 5, 7, 9 or 11.

7. A vector containing the nucleic acid of claim 1, 2, 3 or 4.

8. A host cell containing the vector of claim 7.

9. The host cell of claim 8, wherein the host cell is a bacterial, yeast, insect or mammalian cell.

10. A method of producing a polypeptide, the method comprising culturing the host cell of claim 8 in a culture, expressing the polypeptide encoded by the nucleic acid in the cultured host cell, and isolating the polypeptide from the culture.

11. An isolated polypeptide selected from the group consisting of:
a) a polypeptide consisting of an amino acid sequence which is at least 80% identical to the amino acid sequence of SEQ ID NO:2, 4, 6, 8, 10 or 12;
b) a polypeptide comprising an amino acid sequence which is at least 80% identical to the amino acid sequence of SEQ ID NO:2, 4, 6, 8, 10 or 12;
c) a polypeptide comprising the amino acid sequence of SEQ ID NO:2, 4, 6, 8, 10 or 12 with 0 to 50 conservative amino acid substitutions;
d) a polypeptide which is encoded by a nucleic acid molecule comprising a nucleotide sequence which is at least 80% identical to a nucleic acid comprising the nucleotide sequence of SEQ ID NO:1, 3, 5, 7, 9 or 11; and
e) a naturally occurring allelic variant of a polypeptide comprising the amino acid sequence of SEQ ID NO:2, 4, 6 or 8, wherein the polypeptide is encoded by a nucleic acid molecule which hybridizes to a nucleic acid molecule consisting of SEQ ID NO: 1, 3, 5, 7, 9 or 11, or a complement thereof, under stringent conditions.

12. An isolated polypeptide selected from the group consisting of:
a) the polypeptide encoded by the cDNA insert deposited with ATCC as Accession Number BE300370;
b) the polypeptide encoded by the cDNA insert deposited with ATCC as Accession Number AL520011; and
c) the polypeptide encoded by the cDNA insert deposited with ATCC as Accession Number AL520463.

13. A polypeptide comprising the amino acid sequence of SEQ ID NO:2, 4 , 6, 8, 10 or 12.

14. A polypeptide consisting of the amino acid sequence of SEQ ID NO:2, 4, 6, 8, 10 or 12.

15. The isolated polypeptide of claims 11, 12, 13 or 14, wherein the polypeptide is a phosphatase or a phosphatase inactive mutant.

16. The isolated polypeptide of claim 15, wherein the phosphatase is a serine phosphatase.

17. The isolated polypeptide of claim 16, wherein the serine phosphatase is a small C-terminal domain phosphatase (SCP) that dephosphorylates RNA polymerase II.

18. The isolated polypeptide of claim 15, wherein the serine phosphatase dephosphorylates serine 5 within the C-terminal binding domain (CTD) of RNA polymerase II.

19. The polypeptide of claim 18, wherein the phosphatase is small CTD phosphatase-1 (SCP1), small CTD phosphatase-2 (SCP2), or small CTD phosphatase-3 (SCP3).

20. The isolated polypeptide of claim 11, wherein the amino acid sequence comprises 0 to 30 conservative amino acid substitutions.

21. The isolated polypeptide of claim 11, wherein the amino acid sequence comprises 0 to 10 conservative amino acid substitutions.

22. The isolated polypeptide of claim 11, wherein the amino acid sequence is at least 90% identical to SEQ ID NO:2, 4, 6, 8, 10 or 12.

23. The isolated polypeptide of claim 11, wherein the amino acid sequence is at least 95% identical to SEQ ID NO:2, 4, 6 , 8, 10 or 12.

24. An antibody that selectively binds to a polypeptide of claim 11, 12, 13 or 14.

25. The antibody of claim 24, wherein the antibody is polyclonal or monoclonal.

26. A method of promoting differentiation of a non-neuronal cell in to a cell of the nervous system, the method comprising:
a) contacting the cell with a nucleic acid molecule comprising a nucleic acid sequence encoding a polypeptide selected from the group consisting of SEQ ID NO:10 and SEQ ID NO:12; and
b) expressing the polypeptide in the cell.

27. The method of claim 26, wherein the non-neuronal-cell is a stem cell.

28. The method of claim 26, wherein the stem cell is an embryonic stem cell.

29. The method of claim 26, wherein the cell of the nervous system is a neuron, a sensory neuron, a motoneuron, an interneuron, a glial cell, a microglial cell or an astrocyte.

30. The method of claim 26, wherein the nucleic acid molecule is an expression vector.

31. The method of claim 30, wherein the nucleic acid molecule is a viral genome.

32. A method of inhibiting differentiation of a non-neuronal cell in to a cell of the nervous system, the method comprising:
a) contacting the cell with a nucleic acid molecule comprising a nucleic acid sequence encoding a polypeptide selected from the group consisting of SEQ ID NO:2. SEQ ID NO:4, SEQ ID NO:6 and SEQ ID NO:8; and
b) expressing the polypeptide in the cell.

33. A method of promoting RNA polymerase II associated transcription in a cell, the method comprising:
a) contacting the cell with a nucleic acid molecule comprising a nucleic acid sequence encoding a polypeptide selected from the group consisting of SEQ ID NO:10 and SEQ ID NO:12; and
b) expressing the polypeptide in the cell.

34. A composition comprising an inhibitor of small CTD phosphatase (SCP) gene expression, wherein the inhibitor is selected from the group consisting of:
a) a small molecule inhibitor of gene expression;
b) an anti-sense oligonucleotide; and
c) a small interfering RNA molecule (siRNA or RNAi).

35. The composition of claim 34, wherein the inhibitor of SCP gene expression specifically binds to a polynucleotide selected from the group consisting of:
a) a polynucleotide comprising a sequence selected from the group consisting of SEQ ID NO:1, 3, 5 and 7;
b) a complement of a polynucleotide comprising a sequence selected from the group consisting of SEQ ID NO:1, 3, 5 and 7;
c) a reverse sequence of a polynucleotide comprising a sequence selected from the group consisting of SEQ ID NO:1, 3, 5 and 7;
d) a polynucleotide that encodes a polypeptide comprising a sequence selected from the group consisting of SEQ ID NO:2, 4, 6 and 8;
e) a complement of a polynucleotide that encodes a polypeptide comprising a sequence selected from the group consisting of SEQ ID NO:2, 4, 6 and 8; and
f) a reverse sequence of a polynucleotide that encodes a polypeptide comprising a sequence selected from the group consisting of: SEQ ID NO:2, 4, 6 and 8.

36. The composition of claim 34, wherein the cell is a stem cell.

36. A method of promoting the differentiation of a non-neuronal cell in to a cell of the nervous system, the method comprising contacting the non-neuronal cell with the composition of claim 34 in a sufficient concentration to inhibit the expression of a small CTD phosphatase (SCP).

37. A method of promoting the differentiation of a non-neuronal cell in to a cell of the nervous system, the method comprising contacting the non-neuronal cell with the antibody of claim 24 in a sufficient concentration to inhibit the activity of a small CTD phosphatase (SCP).

38. A method for identifying a compound which modulates the activity of a polypeptide of claim 11, the method comprising:
a) contacting a polypeptide of claim 11 with a test compound; and
b) determining the effect of the test compound on the activity of the polypeptide to thereby identify a compound which modulates the activity of the polypeptide.

39. A method of modulating the differentiation of a mammalian stem cell comprising contacting the stem cell with a compound that modulates SCP1, SCP2 or SCP3 activity, under conditions suitable for differentiation of said stem cell.

40. The method of claim 1, wherein the compound inhibits SCP1, SCP2 or SCP3 activity.

41. A method of transplanting a mammalian stem cell or progenitor cell to a patient in need thereof, the method comprising: (a) contacting the stem cell or progenitor cell with a compound that inhibits SCP1, SCP2 or SCP3 activity to produce a treated stem cell or progenitor cell; and (b) transplanting the treated stem cell into said patient.

42. An in vitro method to modulate the differentiation state of a stem cell, the method comprising: (i) contacting the stem cell with at least one inhibitory RNA molecule (RNAi) comprising a sequence of a gene, or the effective part thereof, selected from the group consisting of SCP1, SCP2 and SCP3; (ii) providing conditions conducive to the growth and differentiation of the cell treated in (i); and optionally (iii) maintaining and/or storing the cell in a differentiated state.


SEQUENCE LISTING (67 sequences)

SEQ ID NO: 1 - DNA, 783 bp, Homo sapiens
atggacagctcggccgtcattactcagatcagcaaggaggaggctcggggcccgctgcggggcaaaggtgaccagaagtcagcagcttcccagaagccccgaagccggggcatcctccactcactcttctgctgtgtctgccgggatgatggggaggccctgcctgctcacagcggggcgcccctgcttgtggaggagaatggcgccatccctaagaccccagtccaatacctgctccctgaggccaaggcccaggactcagacaagatctgcgtggtcatcgacctggacgagaccctggtgcacagctccttcaagccagtgaacaacgcggacttcatcatccctgtggagattgatggggtggtccaccaggtctacgtgttgaagcgtcctcatgtggatgagttcctgcagcgaatgggcgagctctttgaatgtgtgctgttcactgctagcctcgccaagtacgcagacccagtagctgacctgctggacaaatggggggccttccgggcccggctgtttcgagagtcctgcgtcttccaccgggggaactacgtgaaggacctgagccggttgggtcgagacctgcggcgggtgctcatcctggacaattcacctgcctcctatgtcttccatccagacaatgctgtaccggtggcctcgtggtttgacaacatgagtgacacagagctccacgacctcctccccttcttcgagcaactcagccgtgtggacgacgtgtactcagtgctcaggcagccacggccagggagctag

SEQ ID NO: 2 - protein, 260 aa, Homo sapiens
MDSSAVITQISKEEARGPLRGKGDQKSAASQKPRSRGILHSLFCCVCRDDGEALPAHSGAPLLVEENGAIPKTPVQYLLPEAKAQDSDKICVVIDLDETLVHSSFKPVNNADFIIPVEIDGVVHQVYVLKRPHVDEFLQRMGELFECVLFTASLAKYADPVADLLDKWGAFRARLFRESCVFHRGNYVKDLSRLGRDLRRVLILDNSPASYVFHPDNAVPVASWFDNMSDTELHDLLPFFEQLSRVDDVYSVLRQPRPGS

SEQ ID NO: 3 - DNA, 852 bp, Homo sapiens
atggaacacggctccatcatcacccaggcgcggagggaagacgccctggtgctcaccaagcaaggcctggtctccaagtcctctcctaagaagcctcgtggacgtaacatcttcaaggcccttttctgctgttttcgcgcccagcatgttggccagtcaagttcctccactgagctcgctgcgtataaggaggaagcaaacaccattgctaagtcggatctgctccagtgtctccagtaccagttctaccagatcccagggacctgcctgctcccagaggtgacagaggaagatcaaggaaggatctgtgtggtcattgacctcgatgaaacccttgtgcatagctcctttaagccaatcaacaatgctgacttcatagtgcctatagagattgaggggaccactcaccaggtgtatgtgctcaagaggccttatgtggatgagttcctgagacgcatgggggaactctttgaatgtgttctcttcactgccagcctggccaagtatgccgaccctgtgacagacctgctggaccggtgtggggtgttccgggcccgcctattccgtgagtcttgcgtgttccaccagggctgctacgtcaaggacctcagccgcctggggagggacctgagaaagaccctcatcctggacaactcgcctgcttcttacatattccaccccgagaatgcagtgcctgtgcagtcctggtttgatgacatggcagacactgagttgctgaacctgatcccaatctttgaggagctgagcggagcagaggacgtctacaccagccttggggcagctgcgggccccttagcctgccctgcttccaagcgacggccatcccagtag

SEQ ID NO: 4 - protein, 283 aa, Homo sapiens
MEHGSIITQARREDALVLTKQGLVSKSSPKKPRGRNIFKALFCCFRAQHVGQSSSSTELAAYKEEANTIAKSDLLQCLQYQFYQIPGTCLLPEVTEEDQGRICVVIDLDETLVHSSFKPINNADFIVPIEIEGTTHQVYVLKRPYVDEFLRRMGELFECVLFTASLAKYADPVTDLLDRCGVFRARLFRESCVFHQGCYVKDLSRLGRDLRKTLILDNSPASYIFHPENAVPVQSWFDDMADTELLNLIPIFEELSGAEDVYTSLGAAAGPLACPASKRRPSQ

SEQ ID NO: 5 - DNA, 798 bp, Homo sapiens
atggacggcccggccatcatcacccaggtgaccaaccccaaggaggacgagggccggttgccgggcgcgggcgagaaagcctcccagtgcaacgtcagcttaaagaagcagaggagccgcagcatccttagctccttcttctgctgcttccgtgattacaatgtggaggcccctccacccagcagccccagtgtgcttccgccactggtggaggagaatggtgggcttcagaagccaccagctaagtaccttcttccagaggtgacggtgcttgactatggaaagaaatgtgtggtcattgatttagatgaaacattggtgcacagttcgtttaagcctattagtaatgctgattttattgttccggttgaaatcgatggaactatacatcaggtgtatgtgctgaagcggccacatgtggacgagttcctccagaggatggggcagctttttgaatgtgtgctctttactgccagcttggccaagtatgcagaccctgtggctgacctcctagaccgctggggtgtgttccgggcccggctcttcagagaatcatgtgtttttcatcgtgggaactacgtgaaggacctgagtcgccttgggcgggagctgagcaaagtgatcattgttgacaattcccctgcctcatacatcttccatcctgagaatgcagtgcctgtgcagtcctggttcgatgacatgacggacacggagctgctggacctcatccccttctttgagggcctgagccgggaggacgacgtgtacagcatgctgcacagactctgcaataggtag

SEQ ID NO: 6 - protein, 265 aa, Homo sapiens
MDGPAIITQVTNPKEDEGRLPGAGEKASQCNVSLKKQRSRSILSSFFCCFRDYNVEAPPPSSPSVLPPLVEENGGLQKPPAKYLLPEVTVLDYGKKCVVIDLDETLVHSSFKPISNADFIVPVEIDGTIHQVYVLKRPHVDEFLQRMGQLFECVLFTASLAKYADPVADLLDRWGVFRARLFRESCVFHRGNYVKDLSRLGRELSKVIIVDNSPASYIFHPENAVPVQSWFDDMTDTELLDLIPFFEGLSREDDVYSMLHRLCNR

SEQ ID NO: 7 - DNA, 642 bp, Homo sapiens
atgatggggaggccctgcctgctcacagcggggcgcccctgcttgtggaggagaatggcgccatccctaaggcagaccccagtccaatacctgctccctgaggccaaggcccaggactcagacaagatctgcgtggtcatcgacctggacgagaccctggtgcacagctccttcaagccagtgaacaacgcggacttcatcatccctgtggagattgatggggtggtccaccaggtctacgtgttgaagcgtcctcacgtggatgagttcctgcagcgaatgggcgagctctttgaatgtgtgctgttcactgctagcctcgccaagtacgcagacccagtagctgacctgctggacaaatggggggccttccgggcccggctgtttcgagagtcctgcgtcttccaccgggggaactacgtgaaggacctgagccggttgggtcgagacctgcggcgggtgctcatcctggacaattcacctgcctcctatgtcttccatccagacaatgctgtaccggtggcctcgtggtttgacaacatgagtgacacagagctccacgacctcctccccttcttcgagcaactcagccgtgtggacgacgtgtactcagtgctcaggcagccacggccagggagctag

SEQ ID NO: 8 - protein, 213 aa, Homo sapiens
MMGRPCLLTAGRPCLWRRMAPSLRQTPVQYLLPEAKAQDSDKICVVIDLDETLVHSSFKPVNNADFIIPVEIDGVVHQVYVLKRPHVDEFLQRMGELFECVLFTASLAKYADPVADLLDKWGAFRARLFRESCVFHRGNYVKDLSRLGRDLRRVLILDNSPASYVFHPDNAVPVASWFDNMSDTELHDLLPFFEQLSRVDDVYSVLRQPRPGS

SEQ ID NO: 9 - DNA, 783 bp, Drosophila
atggacagctcggccgtcattactcagatcagcaaggaggaggctcggggcccgctgcggggcaaaggtgaccagaagtcagcagcttcccagaagccccgaagccggggcatcctccactcactcttctgctgtgtctgccgggatgatggggaggccctgcctgctcacagcggggcgcccctgcttgtggaggagaatggcgccatccctaagaccccagtccaatacctgctccctgaggccaaggcccaggactcagacaagatctgcgtggtcatcgarctgaacgagaccctggtgcacagctccttcaagccagtgaacaacgcggacttcatcatccctgtggagattgatggggtggtccaccaggtctacgtgttgaagcgtcctcatgtggatgagttcctgcagcgaatgggcgagctctttgaatgtgtgctgttcactgctagcctcgccaagtacgcagacccagtagctgacctgctggacaaatggggggccttccgggcccggctgtttcgagagtcctgcgtcttccaccgggggaactacgtgaaggacctgagccggttgggtcgagacctgcggcgggtgctcatcctggacaattcacctgcctcctatgtcttccatccagacaatgctgtaccggtggcctcgtggtttgacaacatgagtgacacagagctccacgacctcctccccttcttcgagcaactcagccgtgtggacgacgtgtactcagtgctcaggcagccacggccagggagctag

SEQ ID NO: 10 - protein, 260 aa, Drosophila
MDSSAVITQISKEEARGPLRGKGDQKSAASQKPRSRGILHSLFCCVCRDDGEALPAHSGAPLLVEENGAIPKTPVQYLLPEAKAQDSDKICVVIELNETLVHSSFKPVNNADFIIPVEIDGVVHQVYVLKRPHVDEFLQRMGELFECVLFTASLAKYADPVADLLDKWGAFRARLFRESCVFHRGNYVKDLSRLGRDLRRVLILDNSPASYVFHPDNAVPVASWFDNMSDTELHDLLPFFEQLSRVDDVYSVLRQPRPGS

SEQ ID NO: 11 - DNA, 642 bp, Drosophila
atgatggggaggccctgcctgctcacagcggggcgcccctgcttgtggaggagaatggcgccatccctaaggcagaccccagtccaatacctgctccctgaggccaaggcccaggactcagacaagatctgcgtggtcatcgarctgaacgagaccctggtgcacagctccttcaagccagtgaacaacgcggacttcatcatccctgtggagattgatggggtggtccaccaggtctacgtgttgaagcgtcctcacgtggatgagttcctgcagcgaatgggcgagctctttgaatgtgtgctgttcactgctagcctcgccaagtacgcagacccagtagctgacctgctggacaaatggggggccttccgggcccggctgtttcgagagtcctgcgtcttccaccgggggaactacgtgaaggacctgagccggttgggtcgagacctgcggcgggtgctcatcctggacaattcacctgcctcctatgtcttccatccagacaatgctgtaccggtggcctcgtggtttgacaacatgagtgacacagagctccacgacctcctccccttcttcgagcaactcagccgtgtggacgacgtgtactcagtgctcaggcagccacggccagggagctag

SEQ ID NO: 12 - protein, 213 aa, Drosophila
MMGRPCLLTAGRPCLWRRMAPSLRQTPVQYLLPEAKAQDSDKICVVIELNETLVHSSFKPVNNADFIIPVEIDGVVHQVYVLKRPHVDEFLQRMGELFECVLFTASLAKYADPVADLLDKWGAFRARLFRESCVFHRGNYVKDLSRLGRDLRRVLILDNSPASYVFHPDNAVPVASWFDNMSDTELHDLLPFFEQLSRVDDVYSVLRQPRPGS

SEQ ID NO: 13 - DNA, 7020 bp, Drosophila
ctggagcgcggcaggaacccggcccggcccgcctcccagtccgcctagccgcgccggtcccagaagtggcgaaagccgcagccgagtccaggtcacgccgaagccgttgcccttttaagggggagccttgaaacggcgcctgggttccatgtttgcatccgcctcgcgggaaggaaactccatgttgtaacaaagtttcctccgcgccccctccctccccctcccccctagaacctggctcccctcccctccggagctcgcggggatccctccctcccacccctcccctcccccccgcgccccgattccggccccagccgggggggaggccgggcgcccgggccagagtccggccggagcggagcgcgcccggccccatggacagctcggccgtcattactcagatcagcaaggaggaggctcggggcccgctgcggggcaaaggtaccggggctgcggggagggggccgaagccggggcgccgtgggaggagagaaggggccgggatcttccccaggggagccgccgccgccgccccgggcggccgccttagctgtgcccgaagctcccagcccgagagggagcagggagagagtttgaactcagaggaggctcagagacgcggggcggggcctggcgcctttggggcgctcctgtccgctcgaggtgaggaaactgaggcaggaatagagagggaactccttcgggggtttcctggcaggcattgcgtggtgcatgggcgcccccccaccattggcgccaatggggctgtgagatgggggagctgaggagggcgcctatgggccacccgctgagactccgccccaccccccacccccacccccccgggctgcggtccggtagggtcttgggagggggcgccgaggtgacagcaggctggggaggcttggagggatctcccgccaacacacagctacgttccccacaaacttcgcgtcacgcgtggaggcgccgaccccctcggaggcacagagaggacggccggcacttccaagagtcgcttggcgcccgcggggagagtcgtgcgcctagtgggcacgcaccaccccgcaaagcctcgccgccccgacgaggctgcgtcccccagcgtggctgggccggggtgggggggtctgtcttctccttttccccgtgtggacctcaggatctggacgctgcccccaggtctgcccaccctcgcctgggtctggctgccccggaactgagggcaaggtggaaaggctagttgcagggggccggaggggggtggggtgggaggggtatctgtcaatcaggctgctgggctccaggtcggaggtctgggcggggcagggcaaacagatggccactggacactggccccaggccgcgggactgcacccctgcctctgggcccagccgcagtgaggacttcgtacccacgggggtggagaggatggagggagggcaggggtggactgccctgggtcccaggccctggctgtcctgagcaggggtgctcaggtaaggtggggtcaggaggcaccgcaatggggctgatcagcagcagtcatggaggctgtgagaggcagggagagagcaccccaggacctccttctccaggccacgcactccctatgtgggcgccttaatacctgctagacctatttgtctgggagctgcaggagccttggagttgattgtggagccctgacaggggcgtttcagagaaagtcaggagctgccttcgtgtgtctggatgaaggggccacggcaagatcctcctggcccaggggttcacacctgggcacacatgcaggattctgcaggccagtgtgcaccgagcctccaacttgtgcctccctacttcaggtgaccagaagtcagcagcttcccagaagccccgaagccggggcatcctccactcactcttctgctgtgtctgccgggatgatggggaggccctgcctgctcacagcggggcgcccctgcttgtggaggagaatggcgccatccctaaggtgcgtgggggccaggtggggccacgggggcacctggactcagtcttcagggctttaggggaaggggctcctgactgagcttttcaggatggacttgcagacctgaaagtgcagagtaggagggtggcagcctcccctgccaggccctgcccactgtggggaaactgaattctccctcataagtggaagcttttttctaccttggtttttagagaggtctcaaagagccaagaggcctacccaagccctagagctggcaggggcaaagctgggaagggggaagtatctgttcctggggcctggggttcctctggagacggctagggggagaagcctgcgtgggaggaaggaccaggcccggagagaggcaccccagccagccccgccctccctacagcagaccccagtccaatacctgctccctgaggccaaggcccaggactcagacaagatctgcgtggtcatcgacctggacgagaccctggtgcacagctccttcaaggtgggccctgctcaacagccctcagcccgggtctcggggggcatcccccaccctggcctgggagggaggtgtgtgctggaccccatgccctggggctcctcctccaactccagcagctcttttccccccacagccagtgaacaacgcggacttcatcatccctgtggagattgatggggtggtccaccaggtgagggccaggaagaggcagtggtgggcttggcatctgcctccagaccctaggctcttcccaccaatccggagcgcctcggatgggaattggatacatgtggaatgtcagaggcccagagagggtgtgagacttgtcccaaagtcacacagaacctcaagggcttgtgctgactccaagcctgcagagtgggctcctcctctaggctcccccgtgctgtgctccctcgccccaccctgcccgggacccagttcaagtaattcaggataggttgtgtgctgtccagcctgttctccattacttggctcggggaccggtgccctgcagccttggggtgagggggctgcccctggattcctgcactaggctgaggttgaggcaggggaagggattgggaattagggacctcgtgaggtaggactggccagtggagtggaagttttgatcgttttctggcggggggtgggtacagtttccccagcagtggtcagggtagctggccaagcggagcctgcgggcccagtctccttcctgtgcgcctctgcctccctggcccatgccctgccagccctcggccacccccacactgccccactggcccgcagccccctcactggcccgccccccaggtctacgtgttgaagcgtcctcatgtggatgagttcctgcagcgaatgggcgagctctttgaatgtgtgctgttcactgctagcctcgccaaggtgagccccacaggggtcccggggcaaccctgccctcctacctacctcccgcatgcagcccagtgaacctgcgggccccaggatgacccacctcctgctcccagtacgcagacccagtagctgacctgctggacaaatggggggccttccgggcccggctgtttcgagagtcctgcgtcttccaccgggggaactacgtgaaggacctgagccggttgggtcgagacctgcggcgggtgctcatcctggacaattcacctgcctcctatgtcttccatccagacaatgctgtgagtgcgggctggactgggactgggacaggagctgagacccaggaaggggtcagtccattcaggccaccttggcctcttggatccccagttggggggtgggtgccctcccagtccttcctgcattcattgcctgtgcctgccgcccactcccctcatccacctgccctgtagccatatggtcttttcccctcgcacaaagcagagcatctgccatgcacaggggcccccacagggcaacggagtttggaaagtttcaatttttcgaattgccagttgtgacctactgatggcccacagaattaatttagtgggttctgattgggaattttaacaaaatgaaatagaatagaaaatatccggtcgggtgcagtggctcatgcctgtaatcccagcactttgggaagctgaggtgggcaggtagctgagcccagtagttcaagaccagcctcggcaacatagtgaaaccttatgtctacaaaaaatacaaaaactagccaggcgtggtggcgcatgcctggagtcccggctatgcagaaggctgaggtaggagtatcgcttgagccctggaggcagaggctgtggtgagccaagattgtgccactgcactctagcctgggcaacagagcaagaccctgcctcaaaaaaaaaaaaaagtatccaagtgcttcgcacagataaggttaggaattgtgaagcttttgcattgttacgttataaatgtgttttcctggggattgctgtcaaaaaagtttgaacactgtgggtgaggggttttcagaaactgcatgatctgagtagtggctacatagggctggcctggaaattctgcacccaggaccacctgcccccctcatcttcctacacccacttccccaggtaccggtggcctcgtggtttgacaacatgagtgacacagagctccacgacctcctccccttcttcgagcaactcagccgtgtggacgacgtgtactcagtgctcaggcagccacggccagggagctagtgagggtgatggggccaggacctgcccctgaccaatgatacccacacctcctcccaggaagactgcccaggcctttgttaggaaaacccatgggccgccgccacactcagtgccatggggaagcgggcgtctcccccaccagccccaccaggcggtgtaggggcagcaggctgcactgaggaccgtgagctccaggccccgtgtcagtgccttcaaacctcctcccctattctcaggggacctggggggccctgcctgctgctccctttttctgtctctgtccatgctgccatgtttctctgctgccaaattgggccccttggccccttccggttctgcttcctgggggcagggttcctgccttggacccccagtctgggaacggtggacatcaagtgccttgcatagagccccctcttccccgcccagctttcccaggggcacagctctaggctgggaggggagaaccagcccctccccctgccccacctcctcccttgggactgagagggcccctaccaacctttgcctctgccttggagggaggggaggtctgttaccactggggaaggcagcaggagtctgtccttcaggccccacagtgcagcttctccagggccgacagctgagggctgctccctgcatcatccaagcaatgacctcagacttctgccttaaccagccccggggcttggctcccccagctctgagcgtgggggcataggcaggaccccccttgtggtgccatataaatatgtacatgtgtatatagatttttaggggaaggagagagggaagggtcagggtagagacacccctcccttgcccctttcctgggcccagaagttggggggagggagggaaaggatttttacattttttaaactgctattttctgaatggaacaagctgggccaaggggcccaggccctgtcctctgtccctcacacccctttgctccgttcattcattcaaaaaaacatttcttgagcaccttctgtgcccagcatatgctaggcccaccagctaagtgtgtgtggggggtctctacgccagctcatcagtgcctccttgcccatccttcaccggtgcctttgggggatctgtaggaggtgggaccttctgtggggtttggggatctccaggaagcccgaccaagctgtccccttcccctgtgccaacccatctcctacagccccctgcctgatcccctgctggctgggggcagctcccaggatatcctgccttccaactgtttctgaagcccctcctcctaacatggcgattccggaggtcaaggccttgggctctccccagggtctaacggttaaggggacccacataccagtgccaagggggatgtcaagtggtgatgtcgttgtgctcccctcccccagagcgggtgggcggggggtgaatatggttggcctgcatcaggtggccttcccatttaagtgccttctctgtgactgagagccctagtgtgatgagaactaaagagaaagccagacccctatcctgcttctgtggttattgcgggggacttcagcaagtggggtgtgtgccttgcacctgcggctgccgtgggccccccccccgcttcagcacacctagagggctgttggtggagggaggggctgcccggccctcgacacttcaggtgggaagggcagcgtcagagcacaaatttgagcctccaggctgtgctcgtctacgtcttcccgcctcgggtatgtggtctgcaaaatggagatgtgccctattggcaggactaattaagtgcctggacacagacgacaggatactagtagctggaaagcaaaattcgaaggcctgggtaggggcagtcctggaatgcggcgggggagggggcgtggcctctgccctggagcagaggggcggggcttgtgcggctccgaaggcagaggcggggagcggggcgaggctctgggtggaggctccagcggcagaacttgttggcctgggtgcggcgggctccggcgcctggctctgccgggcggcctgggtggggccggcgccggggctcggccccccccgcccctctgcggcctctgagcagccattggccgcgcccccgccccacttcccgccccgccccgcgtccgggaggcacttcctttgcgaaaccgcgcggccccaggcgccggcaggaaatgccctcccgccgtccccagccagcctttgcttgcttcccacgccagccgctagaggcctccctgtcctcgcggacgcaggaactccccgggggctggaaagatggggcccacctcactcacccctttcccgg

SEQ ID NO: 14 - DNA, 4833 bp, Homo sapiens
gccatttcctcctcttgttttcactccggattctccatgttggacccaaactgaggagcccggagctgccgctgggggatcggggccgggggcacccgggggagccgctgcccgggccgcccgccctttgtacaggccgcctcccttcccggtccggggaggaaacgagaggggggatgtgaacagctgtggaagtcggagtctcgggagccggagcgggcccccgcccaggccccccagcccagcccagcccgcgcgcccgcccgtcctcccgtccagccagcccgggcccgcgggattgttagatggaacacggctccatcatcacccaggcgcggagggaagacgccctggtgctcaccaagcaaggcctggtctccaagtcctctcctaagaagcctcgtggacgtaacatcttcaaggcccttttctgctgttttcgcgcccagcatgttggccagtcaagttcctccactgagctcgctgcgtataaggaggaagcaaacaccattgctaagtcggatctgctccagtgtctccagtaccagttctaccagatcccagggacctgcctgctcccagaggtgacagaggaagatcaaggaaggatctgtgtggtcattgacctcgatgaaacccttgtgcatagctcctttaagccaatcaacaatgctgacttcatagtgcctatagagattgaggggaccactcaccaggtgtatgtgctcaagaggccttatgtggatgagttcctgagacgcatgggggaactctttgaatgtgttctcttcactgccagcctggccaagtatgccgaccctgtgacagacctgctggaccggtgtggggtgttccgggcccgcctattccgtgagtcttgcgtgttccaccagggctgctacgtcaaggacctcagccgcctggggagggacctgagaaagaccctcatcctggacaactcgcctgcttcttacatattccaccccgagaatgcagtgcctgtgcagtcctggtttgatgacatggcagacactgagttgctgaacctgatcccaatctttgaggagctgagcggagcagaggacgtctacaccagccttggggcagctgcgggccccttagcctgccctgcttccaagcgacggccatcccagtaggggactttcccacactgtgcctttacgatcagcgtgacagagtagaagctggagtgcctcaccacacggcccggaaacagcgggaagtaactggaaagagctttaggacagcttagatgccgagtgggcgaatgccagaccaatgatacccagagctacctgccgccaacttgttgagatgtgtgtttgactgtgagagagtgtgtgtttgtgtgtgtgttttgccatgaactgtggccccagtgtatagtgtttcagtgggggagaagctgaaagaccaagactcttcccaagttagcttgtctcctctcctgtcaccctaagagccactgagttgtgtagggatgaaractattgaagactccattgccaaaccatggcctttcctcagtgttgtaaggcctatgccaaggataaaggaagggtatgcctttgggtactccaggcatacacctttctgaaatccttctccagccagctgctgcagacaaaagatcacatttctgggaagatgagaacttgtttccagaccagcatccagtggccatcaggtcttgtggcccaaaggctatgcttgcctccggctgagtgcctgggataggccttttctatgtctccccaaggctggggtgctgagcctgccttcctcaccacctagccatagtctcaaacctgtggggaaggaggttttctccctgcccgggaagaggacagataactgatttccgttcttttgactgtgttttaaaattctctttctaaacacagagtgttgggcctggtttgtttctgacaaagttacagtcctgggcctgtaatgaatgtcggcggcgctggggttgcagggaaaagacaaatcctcaaagcgtggacgtgtgtccccatggcttgtggatcagctaagctcgggatcatttccataagtctgcttttcagggattctctgctggtgctggtgcaaggacttctgttccaaaggctgggaaaaactaagctgtcccagcccctcccatttcttgggcagggctcttttcctgttgtgtcttcccccagggcctgtcctgtaccgagctctgtctgttccagcctacatccttcctgggtgttgcttttcctcttaagggcctcagaactcttgctcttcctggggtgagggggaatgagtgttcttgacatgtgacagcctaatgcgcatgctttctgcctctggtaacaggagtgagtgagcccctcagacctgcactctgggtgtctcctgcttacaaaggttcttaatagtgaatgctttaaaattaaagtcatcacgaaatggaagttttcccagggtggaaaataagaggaagtgctgctgtaattgggagcacaaggggcctcccaaaaaggagccccacctcagcatcactgccttaatcgtggcctccctggggtgggtggggttctctcctccctccctccctcctcctggggtgggagggcgctcctgttcccatctctgtgttccctggaggcaggtatcacaaagcatttgtgaattgctttaggtgcagggacaccacccactcaggactcttccccatcatcccttccattgccacaccctagatccagcctcaggaactaacaagttktgagaaaagcaggtggtagagcagcagcttcgtgctctcagcggtggctggctggcatttttctctagcgttgtggtgccaccttcccttcttgtcccaaggttataaggccttgtctttctctttggaatcataaagtggaacagagtccccagaactcatgtgghcatttccgacagcatcactccccggtgcctatggggtcccggtgtacctaaagggagaaggaccccatgtgctagccagaaatatactgtctcttgaaggaaagcaggagctcagactcttagagccagctgtggcttcggacccaaggcctgacctaggctgctatcctaatattggaggaggggcctctcttccaagccccaccctaagggttagcccttggacaaatcttgtgccgtctaggcccagccaggcttttctgactaaataagcaataagaggctctaagctgactgagttgcaaggaccctttccgccctcccttggatctccatgtttctccagatggcggaagagcatgtgccaccccctttcctaacagacttgtccaagtgcttggcgtgggacccatgaccaaagcccaggatggcttggtgggagtgtccctgctgcatctgcatgaagcccctgctttttaggcctcactcccatcagaaccctgcctgcccacctgcaactcccccccaacaatgccattcccacttgccccagagaagctactcggccaaacctagccagggtctgttcttgtggaccagagccagcctagtcattatttgctgtcgggtttccagtttcaccgtgtgttagggtgagggatgattgtaaaatttgctcctcaaaggaatcaggccagactcaattttgggagggcaagacagggaggaggccgcttcatcccagactctcttctagggcttcccaccatcagcccctcccacttgagactggtctttgggaggcaataggccaccatgcctggtcagcaccaattcaagccatgccaggaatctgcctacctgccaggttcagttcttttaaggtgcctcttcagggacacagtgtgtctctctgattgggcttctaaatcaaaagcctgatgttcgtgtccctctcatagggggagctttggacacaggaccagtttggaaaagggtcaggtaagggtttccactctgcacattgtagagggaacactctgtaggcccatgggtcccttactagagaggttgagtgaatttgccttcagttaacatgggaccttctgtttagcttcctcttgcttcccaaagattttaagcattttgtaaatgtataaactcacctctggtaacagtggcccagacgctgctttgtgctaaaagcatgggaaatgtaaaggcagtctttctctgggaaatggatgctattctattctgctgcccctacctgttcctgaggcctcatttagaaagaaaatcccctcagaaggctgtctggcacccagtgtcctagccaggccaagtatatgagaaaggtaagtccattttccccttcaggtcctcagtggattacttaaccactgctgtccctcggtccctttttcctaaacgggtttagttctgtcttttttctccttttttctaaatgctggtaaatatttacattcagccagggaagaggaggccagaggtcgggccagctgccccattcttttaacgttgtagggcctgcccatggagcggaccctcctctttgggcctcgtgagcttttttgcttatcatgttccatttcgtgccgctttcccccttcaagatgccatttggagggtaggggatctgcttcccactgtgactgggctatgggattctgactaccttgcttacagattcatggtttgataaatttgttgtattccaaaacttgaaatgcaggacgccattaagtgtctgtttatatttttggaatatttgtattacttacaattaattaataaaagtgggtttaaaaaacctttccaggaaaaaaaaaaaaaaaaaaaaaaaaaaaaaaaaaaaaa

SEQ ID NO: 15 - DNA, 859 bp, Homo sapiens
atggacggcccggccatcatcacccaggtgaccaaccccaaggaggacgagggccggttgccgggcgcgggcgagaaagcctcccagtgcaacgtcagcttaaagaagcagaggagccgcagcatccttagctccttcttctgctgcttccgtgattacaatgtggaggcccctccacccagcagccccagtgtgcttccgccactggtggaggagaatggtgggcttcagaagccaccagctaagtaccttcttccagaggtgacggtgcttgactatggaaagaaatgtgtggtcattgatttagatgaaacattggtgcacagttcgtttaagcctattagtaatgctgattttattgttccggttgaaatcgatggaactatacatcaggtgtatgtgctgaagcggccacatgtggacgagttcctccagaggatggggcagctttttgaatgtgtgctctttactgccagcttggccaagtatgcagaccctgtggctgacctcctagaccgctggggtgtgttccgggcccggctcttcagagaatcatgtgtttttcatcgtgggaactacgtgaaggacctgagtcgccttgggcgggagctgagcaaagtgatcattgttgacaattcccctgcctcatacatcttccatcctgagaatgcagtgcctgtgcagtcctggttcgatgacatgacggacacggagctgctggacctcatccccttctttgagggcctgagccgggaggacgacgtgtacagcatgctgcacagactctgcaataggtagccctggcctctgcctgcctcccgcctgtgcactctggaacctctggcctcaggggacctgc

SEQ ID NO: 16 - DNA, 754 bp, Homo sapiens
atgatggggaggccctgcctgctcacagcggggcgcccctgcttgtggaggagaatggcgccatccctaaggcagaccccagtccaatacctgctccctgaggccaaggcccaggactcagacaagatctgcgtggtcatcgacctggacgagaccctggtgcacagctccttcaagccagtgaacaacgcggacttcatcatccctgtggagattgatggggtggtccaccaggtctacgtgttgaagcgtcctcacgtggatgagttcctgcagcgaatgggcgagctctttgaatgtgtgctgttcactgctagcctcgccaagtacgcagacccagtagctgacctgctggacaaatggggggccttccgggcccggctgtttcgagagtcctgcgtcttccaccgggggaactacgtgaaggacctgagccggttgggtcgagacctgcggcgggtgctcatcctggacaattcacctgcctcctatgtcttccatccagacaatgctgtaccggtggcctcgtggtttgacaacatgagtgacacagagctccacgacctcctccccttcttcgagcaactcagccgtgtggacgacgtgtactcagtgctcaggcagccacggccagggagctagtgagggtgatggggccaggacctgcccctgaccaatgatacccacacctcctcccaggaagactgcccaggcctttgttaggaaaacccatgggccgccgccacactcagtg

SEQ ID NO: 17 - DNA, 27 bp, Artificial sequence
  Synthetic construct polymerase binding site
gaattaatacgactcactatagggaga

SEQ ID NO: 18 - DNA, 25 bp, Artificial sequence
  Primer
atgggcgaactatacgagtgcgttc

SEQ ID NO: 19 - DNA, 25 bp, Artificial sequence
  Primer
atcaacgacaacttcgagatcgtcg

SEQ ID NO: 20 - DNA, 24 bp, Artificial sequence
  Primer
atgtcgctcttgcaaaaactaagc

SEQ ID NO: 21 - DNA, 25 bp, Artificial sequence
  Primer
tgaagatcctcaccgagcgcggcta

SEQ ID NO: 22 - DNA, 25 bp, Artificial sequence
  Primer
cagctggtgcgggagtacggcttcc

SEQ ID NO: 23 - DNA, 22 bp, Artificial sequence
  Primer
gagctgtcgttgagctttggcg

SEQ ID NO: 24 - DNA, 23 bp, Artificial sequence
  Primer
actgggcctattactactggctc

SEQ ID NO: 25 - DNA, 25 bp, Artificial sequence
  Primer
caacgaagccgagcgagccatccag

SEQ ID NO: 26 - DNA, 26 bp, Artificial sequence
  Primer
gcaacaactgggccaagggtcattac

SEQ ID NO: 27 - DNA, 26 bp, Artificial sequence
  Primer
gccttccaagagcacgacgtacaaag

SEQ ID NO: 28 - DNA, 26 bp, Artificial sequence
  Primer
ctcgccaatcaagtaccttgtgctgc

SEQ ID NO: 29 - DNA, 25 bp, Artificial sequence
  Primer
cttcgctcgcacctcagaaacgatc

SEQ ID NO: 30 - DNA, 25 bp, Artificial sequence
  Primer
caacggaactaacggccgctccgag

SEQ ID NO: 31 - DNA, 22 bp, Artificial sequence
  Primer
ctcgccattgttctcctggtgg

SEQ ID NO: 32 - DNA, 25 bp, Artificial sequence
  Primer
cttgtctgctgctggttcaacatgg

SEQ ID NO: 33 - DNA, 25 bp, Artificial sequence
  Primer
gcggttggagtagccaaactcgttg

SEQ ID NO: 34 - DNA, 25 bp, Artificial sequence
  Primer
ttataggatatcttcgattttcggc

SEQ ID NO: 35 - DNA, 26 bp, Artificial sequence
  Primer
gaccggactcgtcatactcctgcttg

SEQ ID NO: 36 - DNA, 25 bp, Artificial sequence
  Primer
tgcgcagctcgcccatgtagacctg

SEQ ID NO: 37 - DNA, 23 bp, Artificial sequence
  Primer
cgcgtggattggggaagaaggtc

SEQ ID NO: 38 - DNA, 24 bp, Artificial sequence
  Primer
ccgtaaaaccgcgcgcattaaagt

SEQ ID NO: 39 - DNA, 24 bp, Artificial sequence
  Primer
tggtcatggtcacgaatccgaatc

SEQ ID NO: 40 - DNA, 27 bp, Artificial sequence
  Primer
cttggcatcgaacatctgctgggtcag

SEQ ID NO: 41 - DNA, 26 bp, Artificial sequence
  Primer
cgatcagaagtggatcgcggtcctta

SEQ ID NO: 42 - DNA, 24 bp, Artificial sequence
  Primer
ccctggctgaagcagaacttcatg

SEQ ID NO: 43 - DNA, 25 bp, Artificial sequence
  Primer
tatggcataaaaggtgtggccattc

SEQ ID NO: 44 - DNA, 24 bp, Artificial sequence
  Primer
gttctcgccatcgttgagatctgc

SEQ ID NO: 45 - DNA, 22 bp, Artificial sequence
  Primer
cgtacatgaggtagaccctgga

SEQ ID NO: 46 - DNA, 27 bp, Artificial sequence
  Primer
cggccgtcattactcagatcagcaagg

SEQ ID NO: 47 - DNA, 22 bp, Artificial sequence
  Primer
tccaccaccctgtgttgctgta

SEQ ID NO: 48 - DNA, 27 bp, Artificial sequence
  Primer
catctctgatctcgactgctccagcag

SEQ ID NO: 49 - DNA, 30 bp, Artificial sequence
  Primer
tgccctcacccaaggtctctgacactgtgg

SEQ ID NO: 50 - DNA, 30 bp, Artificial sequence
  Primer
ctgtggccatggagggaaacagtggcttcc

SEQ ID NO: 51 - DNA, 28 bp, Artificial sequence
  Primer
gcaaccgcaggcacgactgtttacggag

SEQ ID NO: 52 - DNA, 29 bp, Artificial sequence
  Primer
ccatcgcctgcgaaacctccccaggtaga

SEQ ID NO: 53 - DNA, 28 bp, Artificial sequence
  Primer
gcagtgaacagcacacattcaaagagct

SEQ ID NO: 54 - DNA, 20 bp, Artificial sequence
  Primer
accacagtccatgccatcac

SEQ ID NO: 55 - DNA, 27 bp, Artificial sequence
  Primer
gggtcagagagtggtgatgccacagtg

SEQ ID NO: 56 - DNA, 28 bp, Artificial sequence
  Primer
cttgaacagctcctggatggcagtgctg

SEQ ID NO: 57 - DNA, 28 bp, Artificial sequence
  Primer
agaagtccaggagcagctgagggagcac

SEQ ID NO: 58 - DNA, 30 bp, Artificial sequence
  Primer
agatgaccatccggaagaagttggccttgt

SEQ ID NO: 59 - DNA, 30 bp, Artificial sequence
  Primer
agccaactcagctggactctctccagcttc

SEQ ID NO: 60 - DNA, 30 bp, Artificial sequence
  Primer
tgcggtttatattatcctgcacgccgggag

SEQ ID NO: 61 - DNA, 27 bp, Artificial sequence
  Primer
ggagccctatgcagggtaagggaataa

SEQ ID NO: 62 - DNA, 28 bp, Artificial sequence
  Primer
aactatttctgggtcactccttagacac

SEQ ID NO: 63 - DNA, 30 bp, Artificial sequence
  Primer
ctggataagttactgaagagtgggctttgg

SEQ ID NO: 64 - DNA, 29 bp, Artificial sequence
  Primer
caccggttcgagtccccggagaggatatc

SEQ ID NO: 65 - DNA, 28 bp, Artificial sequence
  Primer
gggctttgatttttggagccaccttgtg

SEQ ID NO: 66 - DNA, 29 bp, Artificial sequence
  Primer
gctgggaggaatgctttctaatgcatttg

SEQ ID NO: 67 - DNA, 25 bp, Artificial sequence
  Primer
cagacgacaagttacatgcaacatg